SEQUENCE LISTING (4 sequences)

SEQ ID NO: 1 - protein, 32 aa, Artificial Sequence
  diuretic or natriuretic peptide
TAPRSLRRSSCFGLKLDRIGSMSGLGCNSFRY

SEQ ID NO: 2 - protein, 37 aa, Artificial Sequence
  diuretic or natriuretic peptide
GLSKGCFGLKLDRIGSMSGLGCPSLRDPRPNAPSTSA

SEQ ID NO: 3 - protein, 37 aa, Artificial Sequence
  diuretic or natriuretic peptide
GLSKGCFGRKMDRIGSMSGLGCPSLRDPRPNAPSTSA

SEQ ID NO: 4 - protein, 37 aa, Artificial Sequence
  diuretic or natriuretic peptide
GLSKGCFGLKLDRISSSSGLGCPSLRDPRPNAPSTSA

(19) United States
(12) Patent Application Publication
(10) Pub. No.: US 2014/0031787 A1
(43) Pub. Date: Jan. 30, 2014

(54) FEEDBACK-BASED DIURETIC OR NATRIURETIC MOLECULE ADMINISTRATION

(71) Applicants: John Burnes, Coon Rapids, MN (US); VenKatesh Manda, Stillwater, MN (US); William Van Antwerp, Valencia, CA (US); Jamie Williams, St. Louis Park, MN (US)

(72) Inventors: John Burnes, Coon Rapids, MN (US); VenKatesh Manda, Stillwater, MN (US); William Van Antwerp, Valencia, CA (US); Jamie Williams, St. Louis Park, MN (US)

(73) Assignee: Medtronic, Inc., Minneapolis, MN (US)

(21) Appl. No.: 13/844,549

(22) Filed: Mar. 15, 2013

Related U.S. Application Data
(60) Provisional application No. 61/623,226, filed on Apr. 12, 2012.

(51) Int. Cl. A61M 5/172

(52) U.S. Cl. CPC A61M 5/1723
USPC 604/505; 604/66; 604/503

(57) ABSTRACT

Devices, systems and methods using feedback from sensors for the treatment of pathological conditions such as Kidney Disease (KD) alone, Heart Failure (HF) alone, KD with concomitant HF or cardiorenal diseases syndrome (CRS) are described. The devices, systems and methods monitor and gather patient information and administer one or more diuretic or natriuretic molecules.

### FIELD OF THE INVENTION

The invention relates to devices, systems and methods for the treatment of pathological conditions such as Kidney Disease (KD) alone, Heart Failure (HF) alone, KD with concomitant HF or cardiorenal syndrome (CRS) using feedback from sensors. The devices, systems and methods of the invention can increase or decrease in vivo levels of one or more diuretic or natriuretic molecules in the body of a subject to regulate and or control the outcome of a therapeutic regimen(s) using feedback obtained from the sensors. In addition to monitoring and gathering patient information, the devices, systems and methods administer one or more diuretic or natriuretic molecules through any number of routes of administration, including but not limited to, subcutaneous, intravascular, intraperitoneal, intradermal and direct to organ diuretic or natriuretic molecules. The systems and methods for controlling in vivo levels of one or more diuretic or natriuretic molecules include, but are not limited to, implanted and external pumps, depot injection, direct delivery catheter systems, single all-in-one implanted device with sensors, and/or local controlled release technology.

### BACKGROUND

The physiological state of a patient can affect the delivery of a drug to renal tissue. Many patients having acute HF, KD or hypotension exhibit a reduced response to systemically administered drugs due to non-ideal hemodynamic factors that reduce the amount of drug reaching the kidney tissues due to reduced cardiac output and/or renal perfusion. Ideal dosing of diuretic or natriuretic molecules can therefore be difficult to achieve despite knowing a patient's physiologic parameters useful for prescription such as weight or extent of kidney disease and/or heart failure, since all factors that can affect the transportation of diuretic or natriuretic molecules to the kidneys are not known.

There are also uncertainties involved in treating a patient with systemically administered diuretic or natriuretic molecules. For example, many separate pathways may exist for metabolizing and eliminating a drug from the body due to the ubiquity of agents that can act on small peptides. The presence of endogenous proteolytic enzymes can quickly metabolize many peptides at most routes of administration. A small peptide can be readily degraded by peptidases and other enzymes present in the body. As such, the response to a specific administration regimen of a natriuretic molecule can vary between individuals who may otherwise share descriptive characteristics such as sex, weight, BMI, kidney function and other classification parameters used to determine an appropriate administration regimen. Similarly, some diuretics may cause a substantial diuresis—up to 20% of the filtered load of NaCl and water. The relative diuresis is large when compared to normal renal sodium reabsorption which leaves only ˜0.4% of filtered sodium in the urine, hence demonstrating a clear need to closely monitor such powerful diuretics.

The effectiveness of a drug delivered through various routes of administration can also be affected by humoral and/or hemodynamic effects that direct the blood flow to or away from the tissue or organ system that is targeted by a drug. For example, a reduction in renal perfusion pressure caused by a drop in blood pressure can limit the effectiveness of some drugs targeting the renal tissues presumably due to the hemodynamics that direct systemically-administered drugs away from the kidneys (Redfield M. et al., Restoration of renal response to atrial natriuretic factor in experimental low-output heat failure. Am. J. Physiol. 257:R917-23 (1989)). In addition, peptides and proteins are generally hydrophilic, do not readily penetrate lipophilic biomembranes and have short biological half-lives due to rapid metabolism and clearance. These factors result in significant variability among patients and deter the effective and efficient use of protein drug therapies. As such, significant variations in response to natriuretic molecules are possible where changes in cardiac function, kidney function or fluid status may change during a treatment period for a particular patient. Similarly, many drugs can quickly cause a patient to become hypoosmotic.

Hence, there is an unmet need for drug delivery systems and device-mediated methods of natriuretic molecule delivery that offer significant advantages over conventional delivery systems by providing increased efficiency and improved performance in obtaining, regulating and/or controlling dosing of diuretic or natriuretic molecules via active control of a therapeutic regimen.

There is also a need for monitoring the condition of the patient to obtain patient parameters that can be used to adjust a therapeutic profile and/or regimen, and optionally determine a personalized program or algorithm for delivery of one or more natriuretic patients based on sensor feedback from a particular patient. There is a need for systems and methods to provide feedback data on patient parameters to automatically diagnose the precise individualized therapeutic regimen for a specific patient to make any necessary adjustments based on a patient's actual response to a particular dosing therapy. There is a need for systems and methods capable of monitoring patient parameters in order to diagnose the condition of the patient prior to, during and after therapeutic delivery of diuretic or natriuretic molecules.

There is also an unmet need for electronically monitoring and/or collecting data from the patient and/or maintaining a set of patient data obtained from feedback sensors to identify and/or to assist in making a clinical decision or making a modification to a patient treatment profile.

### SUMMARY OF THE INVENTION

The disclosure is directed to systems and methods for delivery of one or more diuretic or natriuretic molecules to a patient having Kidney Disease (KD) alone, Heart Failure (HF) alone, KD with concomitant HF, or cardiorenal syndrome (CRS) via any administration route including but not limited to continuous subcutaneous (SQ) administration via open or closed loop control. The systems and methods can be used to maintain in vivo concentrations of one or more diuretic or natriuretic molecules above a critical efficacy threshold for an extended period of time by monitoring and actively adjusting the delivery of the diuretic or natriuretic molecules. Both bolus and continuous SQ delivery of diuretic or natriuretic molecules are contemplated.

The invention contemplates a medical device having one or more sensors adapted to detect at least one physiologic parameter relating to any one of cardiac function, kidney function or fluid status of a patient, a pump for delivering one or more diuretic or natriuretic molecules to a patient, wherein the pump is controlled by a control system, wherein the control system applies an algorithm to data received from the one or more sensors, and the algorithm determines the need for the patient to have an increased or decreased amount of a diuretic or natriuretic molecules. The control system has a data aggregation device function or has access to a data aggregation device function for receiving and storing data from the one or more sensors. The invention contemplates one or more sensors to determine a physiological parameter selected from blood pressure, pulmonary artery pressure, left atrial pressure, central venous pressure, lung fluid volume, proteinuria, plasma renin, central venous pressure, right atrial pressure, cardiac output, and GFR. The data aggregation device communicates with the one or more sensors, such as by Bluetooth or 802.11 protocols, or through a wired device input/output port. The medical device also has a local monitor, a cell phone or a cellular device that receives data from the one or more sensors, and has a data aggregation device function remote from patient and the local monitor, cell phone or cellular device in the vicinity of the patient, wherein the local monitor, cell phone or cellular device relays data from the one or more sensors to a device having the data aggregation device function. The medical device relays data using a CDMA or GSM cellular network, the Internet or a wired phone network.

The data aggregation device function and the control system can be co-located. The protocol is adjusted in an iterative process to improve the fluid status, cardiac function or kidney function of the patient while maintaining the cardiovascular stability of the patient. The sensor measures one or more selected from pulmonary artery pressure, right atrial pressure, intrathoracic impedance and peripheral edema to provide an indication of the fluid status of the patient. Additional sensors measure one or more patient parameters selected from blood pressure and central venous pressure.

The invention further contemplates a system having a sensor that generates an electrical signal that varies as function of a parameter associated with at least one physiologic parameter relating to any one of cardiac function, kidney function or fluid status of a patient. A processor that processes the electrical signal to detect a change in the cardiac function, kidney function or fluid status of a patient, and a pump controlled by the processor for administering one or more diuretic or natriuretic molecules to the patient based upon the detected change in the cardiac function, kidney function or fluid status of the patient is further contemplated.

The invention contemplates a method having steps of obtaining data from one or more sensors configured to measure one or more physiological parameters of a patient, applying an algorithm to the data received from the one or more sensors, wherein the algorithm determines the need for the patient to have an increased or decreased amount of a diuretic or natriuretic molecules, and performing a treatment selected from at least one of: a) administering one or more diuretic or natriuretic molecules at a rate determined by applying the algorithm, and b) stopping the administration of diuretic or natriuretic molecules as determined by applying the algorithm. The administration of the one or more diuretic or natriuretic molecules is performed by a pump for administering the diuretic or natriuretic molecules wherein the pump is controlled by a control system for receiving data from the one or more sensors either directly or indirectly through a separate processor that communicates with the pump.

The invention contemplates a method wherein the pump is one or more selected from an implantable pump implanted in the patient and an external pump. The physiological parameters are selected from the group consisting of blood pressure, pulmonary artery pressure, left atrial pressure, central venous pressure, lung fluid volume, proteinuria, plasma renin, central venous pressure, right atrial pressure, cardiac output, and GFR.

The present invention in one or more embodiments provides a medical device which includes: one or more sensors adapted to detect at least one physiologic parameter relating to any one of cardiac function, kidney function or fluid status of a patient, a pump for delivering one or more diuretic or natriuretic molecules to a patient, and an algorithm for determining the need for the patient to have an increased or decreased amount of a diuretic or natriuretic molecule.

The pump of the medical device may be controlled by a control system, the control system applying the algorithm to data received from the one or more sensors.

The control system of the medical device may have a data aggregation device function or has access to a data aggregation device function for receiving and storing data from the one or more sensors.

The one or more sensors of the medical device may determine a physiological parameter selected from blood pressure, pulmonary artery pressure, left atrial pressure, central venous pressure, lung fluid volume, proteinuria, plasma renin, central venous pressure, right atrial pressure, cardiac output, and GFR.

The data aggregation device function of the medical device may communicate with the one or more sensors by Bluetooth or 802.11 protocols or through a wired device input/output port.

The medical device may further include one or more selected from a local monitor, a cell phone or a cellular device that receives data from the one or more sensors.

A device having the data aggregation device function may be remote from patient and the local monitor, cell phone or cellular device is in the vicinity of the patient, wherein the local monitor, cell phone or cellular device relays data from the one or more sensors to a device having the data aggregation device function.

The data relayed from the local monitor, cell phone or cellular device may use a CDMA or GSM cellular network, the Internet or a wired phone network.

The data aggregation device function and the control system of the medical device may be co-located.

In relation to the medical device, a protocol may be adjusted in an iterative process to improve the fluid status, cardiac function or kidney function of the patient while maintaining the cardiovascular stability of the patient.

In relation to the medical device, a sensor may measure one or more selected from pulmonary artery pressure, right atrial pressure, intrathoracic impedance and peripheral edema to provide an indication of the fluid status of the patient.

In relation to the medical device, an additional sensor may measure one or more selected from blood pressure and central venous pressure.

In relation to the medical device, the one or more natriuretic molecules may be selected from the group consisting of long-acting natriuretic peptide (LANP), kaliuretic peptide (KP), urodilatin (URO), atrial natriuretic peptide (ANP), brain natriuretic peptide (BNP), vessel dilator (VD) and chimeric natriuretic peptides including peptides selected from CH-NP, CU-NP, and BD-NP.

In certain embodiments, a medical device decreases a rate of administration of a pump when a physiological parameter shows a value that indicates an improvement in cardiac function, kidney function or fluid status of the patient compared to a prior value.

In certain embodiments, a medical device stops administration by a pump when the physiological parameter improves to reach a targeted level.

In certain embodiments, a medical device increases a rate of administration of a pump when a physiological parameter shows a value that indicates a worsening in cardiac function, kidney function or fluid status of the patient compared to a prior value.

In certain embodiments, a medical device issues an alert when a physiological parameter has a value that indicates a worsening in cardiac function, kidney function or fluid status of the patient and the value has reached a critical level.

In certain embodiments, a medical device increases a rate of administration of a pump when a first physiological parameter has a first value that is at or above a predetermined range and a second physiological parameter has a second value that indicates a worsening in cardiac function, kidney function or fluid status of the patient compared to a prior value.

In certain embodiments, a medical device issues an alert when a first physiological parameter has a first value that is at or above a predetermined range and a second physiological parameter has a second value that indicates a worsening in cardiac function, kidney function or fluid status of the patient compared to a prior value and a rate of administration of the pump is at a maximum level.

In certain embodiments, a medical device maintains a rate of administration of a pump when a first physiological parameter has a first value that is at or above a predetermined range and a second physiological parameter has a second value that indicates an improvement in cardiac function, kidney function or fluid status of the patient compared to a prior value.

In certain embodiments, a medical device issues an alert when a first physiological parameter is at a critically low value.

In certain embodiments, a medical device decreases a rate of administration of a pump when a first physiological parameter has a first value that is below a predetermined range and a second physiological parameter has a second value that indicates an improvement in cardiac function, kidney function or fluid status of the patient compared to a prior value.

In certain embodiments, a medical device maintains a rate of administration of a pump when a first physiological parameter has a first value that is below a predetermined range and a second physiological parameter has a second value that indicates stability or worsening in cardiac function, kidney function or fluid status of the patient compared to a prior value.

In certain embodiments, a medical device basis a decision to adjust a pump rate based at least in part upon a first physiological parameter that is systolic blood pressure and a second physiological parameter that is fluid volume or fluid status of the patient.

In certain embodiments, a medical device can set and adjust a maximum rate of administration of a pump.

In certain embodiments, a medical device increases a maximum rate of administration of a pump by an incremental amount when all of the following are present: 1) the medical device has operated the pump at a preliminary maximum dose, where the preliminary maximum dose is a present maximum rate of administration of the pump, 2) the medical device has operated the pump at the preliminary maximum rate of administration for at least a predetermined period of time, and 3) one or more physiological parameters of the patient has been stable over the predetermined period of time to indicate that homeostasis or near homeostasis of the physiological parameter is present.

In certain embodiments, a medical device cannot increase the maximum rate of administration of a pump above a predetermined limit.

In certain embodiments, a medical device initiates delivery of a first diuretic or a second diuretic prior to initiating delivery of a natriuretic molecule or peptide to the patient.

In certain embodiments, a medical device initiates delivery of the a diuretic when 1) a blood pressure physiological parameter of the patient is above a predetermined rage or the blood pressure physiological parameter is below a predetermined range and a serum creatinine physiological parameter of the patient is within a predetermined range, and 2) a blood serum potassium concentration of the patient is above a predetermined range.

In certain embodiments, a medical device initiates delivery of a second diuretic when 1) a blood pressure physiological parameter of the patient is above a predetermined rage or the blood pressure physiological parameter is below a predetermined range and a serum creatinine physiological parameter of the patient is within a predetermined range, and 2) a blood serum potassium concentration of the patient is within a predetermined range.

In certain embodiments, a first diuretic is a loop diuretic.

In certain embodiments, a second diuretic is a calcium sparing diuretic or a loop diuretic and calcium.

In certain embodiments, a medical device initiates an alert when a blood pressure physiological parameter is below a predetermined range a serum creatinine physiological parameter of the patient is above a predetermined range.

The present invention in one or more embodiments further provides a system which includes: a sensor that generates an electrical signal that varies as function of a parameter associated with least one physiologic parameter relating to any one of cardiac function, kidney function or fluid status of a patient; a processor that processes the electrical signal to detect a change in the cardiac function, kidney function or fluid status of a patient; and a pump controlled by the processor for administering one or more diuretic or natriuretic molecules to the patient based upon the detected change in the cardiac function, kidney function or fluid status of the patient.

The one or more sensor of the system may determine a physiological parameter selected from blood pressure, pulmonary artery pressure, left atrial pressure, central venous pressure, lung fluid volume, proteinuria, plasma renin, central venous pressure, right atrial pressure, cardiac output, and GFR.

The processor of the system may have a data aggregation device function or has access to a data aggregation device function for receiving and storing data from the one or more sensors.

The processor may communicate with the one or more sensors by Bluetooth or 802.11 protocols or through a wired device input/output port.

The processor may be remote from the patient and a local monitor, cell phone or cellular device is in the vicinity of the patient, wherein the local monitor, cell phone or cellular device relays data from the one or more sensors to the processor.

In relation to the system, the data is relayed using a CDMA or GSM cellular network, the Internet or a wired phone network.

In relation to the system, the data aggregation device function and the control system may be co-located.

In relation to the system, a protocol for controlling the pump may be adjusted in an iterative process to improve the fluid status, cardiac function or kidney function of the patient while maintaining the cardiovascular stability of the patient.

In relation to the system, a sensor may generate electrical signals that vary based on one or more selected from pulmonary artery pressure, right atrial pressure, intrathoracic impedance and peripheral edema to provide an indication of the fluid status of the patient.

In relation to the system, an additional sensor may generate electrical signals that vary based on one or more selected from blood pressure and central venous pressure.

In relation to the system, the one or more natriuretic molecules may be selected from the group consisting of long-acting natriuretic peptide (LANP), kaliuretic peptide (KP), urodilatin (URO), atrial natriuretic peptide (ANP), brain natriuretic peptide (BNP), vessel dilator (VD), and chimeric natriuretic peptides including peptides selected from CH-NP, CU-NP, and BD-NP.

The present invention in one or more embodiments further provides a method which includes the steps of: obtaining data from one or more sensors configured to measure one or more physiological parameters of a patient; applying an algorithm to the data received from the one or more sensors, the algorithm determining the need for the patient to have an increased or decreased amount of a diuretic or natriuretic molecules; and performing a treatment selected from at least one of: a) administering one or more diuretic or natriuretic molecules at a rate determined by applying the algorithm, and b) stopping the administration of diuretic or natriuretic molecules as determined by applying the algorithm, wherein the administration of the one or more diuretic or natriuretic molecules is performed by a pump for administering the diuretic or natriuretic molecules, the pump controlled by a control system for receiving data from the one or more sensors.

In relation to the method, the pump may be one or more selected from an implantable pump implanted in the patient and an external pump.

In relation to the method, the one or more physiological parameters may be selected from the group consisting of blood pressure, pulmonary artery pressure, left atrial pressure, central venous pressure, lung fluid volume, proteinuria, plasma renin, central venous pressure, right atrial pressure, cardiac output, and GFR.

In relation to the method, a rate of administration of the diuretic or natriuretic molecules may be increased if a physiological parameter measured by the one or more sensors indicates a deterioration in the fluid status, cardiac function or kidney function of the patient.

In relation to the method, a rate of administration of the diuretic or natriuretic molecules may be decreased if a physiological parameter measured by the one or more sensors indicates a deterioration in the fluid status, cardiac function or kidney function of the patient.

In relation to the method, a rate of administration of the natriuretic may be adjusted based upon at least a first physiological parameter and a second physiological parameter, wherein the first physiological parameter indicates the cardiovascular stability of the patient and the second physiological parameter indicates the fluid status, cardiac function or kidney function of the patient.

In relation to the method, the rate of administration of the diuretic or natriuretic molecules may be increased if the first physiological parameter indicates that the patient has cardiovascular stability and the second physiological parameter indicates deterioration in the fluid status, cardiac function or kidney function of the patient.

In relation to the method, the rate of administration of the diuretic or natriuretic molecules may be decreased if the first physiological parameter is below a threshold and the second physiological parameter indicates improvement in the fluid status, cardiac function or kidney function of the patient.

In relation to the method, the data from the one or more sensors may be received by a device acting as a data aggregation device for storing data received from the one or more sensors, and the data stored in the data aggregation device is accessible by the control system for controlling operation of a pump for administering the diuretic or natriuretic molecules.

In relation to the method, the data aggregation device and the control system may be co-located.

In relation to the method, the patient may have heart failure, kidney disease, heart failure with concomitant kidney disease, or cardiorenal syndrome.

In relation to the method, the one or more natriuretic molecules may be selected from the group consisting of long-acting natriuretic peptide (LANP), kaliuretic peptide (KP), urodilatin (URO), atrial natriuretic peptide (ANP), brain natriuretic peptide (BNP), vessel dilator (VD), and chimeric natriuretic peptides including peptides selected from CH-NP, CU-NP, and BD-NP.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 shows a representation of fluid and blood pressure homeostasis.

FIG. 2 shows a schematic of a system for managing data obtained from one or more sensors in accordance with some embodiments.

FIG. 3 shows a schematic for a data aggregation device (DAD) in accordance with some embodiments of the invention.

FIG. 4 illustrates channels for the data aggregation device to communicate with a control system in accordance with some embodiments of the invention.

FIG. 5 illustrates the relay of data to and from the data aggregation device and a control system in accordance with some embodiments of the invention.

FIG. 6 illustrates the relay of data to and from the data aggregation device and one or more sensors in accordance with some embodiments.

FIG. 7 illustrates a methodology for controlling administration of diuretic or natriuretic molecules in accordance with some embodiments of the invention.

FIG. 8 illustrates a methodology for controlling administration of a diuretic or natriuretic molecule in accordance with some embodiments of the invention.

FIG. 9 illustrates a methodology for adjusting a maximum limit for dosing a diuretic or natriuretic molecule in accordance with some embodiments of the invention.

FIG. 10 illustrates a methodology for initiating dosing of diuretic or natriuretic molecules in accordance with some embodiments.

FIG. 11 illustrates an embodiment of a local monitor which includes a sensor and an implantable Cardiac Monitor.

FIG. 12 illustrates an embodiment of a local monitor which includes a sensor, a wired external blood pressure, and heart rate monitor.

FIG. 13 illustrates an embodiment of a local monitor which includes a sensor and a wired external health monitor.

FIG. 14 depicts an implant monitor which provides an in-vivo assessment of the fluid pressure and temperature around the monitor that is positioned in a living being.

FIG. 15 depicts a noninvasive cardiac output monitoring (NICOM) monitor which provides a method to measure cardiac data for a human patient.

FIG. 16 shows right atrial pressures for a canine heart failure model treated with a natriuretic peptide.

FIG. 17 shows pulmonary capillary wedge pressures for a canine heart failure model treated with a natriuretic peptide.

FIG. 18 shows GFR for a canine heart failure model treated with a natriuretic peptide.

FIG. 19A and FIG. 19B show blood pressure for an animal model treated with a natriuretic peptide.

FIG. 20A shows mean systolic blood pressure observed during a 24-hour period of CD-NP subcutaneous infusion in subjects to a clinical study and in a six-hour post-infusion period. FIG. 20B shows mean diastolic blood pressure observed during a 24-hour period of CD-NP subcutaneous infusion in subjects to a clinical study and in a six-hour post-infusion period.

FIG. 21 shows the effect of a chimeric natriuretic peptide administered by subcutaneous infusion on blood pressure in an animal model.

FIG. 22 shows the effect of a chimeric natriuretic peptide administered by subcutaneous infusion on albumin excretion in an animal model.

FIG. 23 depicts the cardiac data measured by a noninvasive cardiac output monitoring (NICOM) monitor.

FIG. 24 depicts the cardiac data in response to sequential fluid boluses.

FIG. 25 depicts the increase in cardiac output data produced by incremental infusion doses of dobutamine.

FIG. 26 depicts the cardiodepressant effect of esmolol given after dobutamine and recalibration.

FIG. 27 depicts the response to increasing blood pressure with phenylephrine followed by decreasing blood pressure with sodium nitroprusside (SNP).

### DETAILED DESCRIPTION

The medical devices and systems of the invention contain a drug provisioning component to administer a therapeutically effective amount of one or more diuretic or natriuretic molecules to a subject suffering from CKD alone, HF alone, CKD with concomitant HF or cardiorenal syndrome (CRS)e wherein the drug provisioning component maintains a plasma concentration of one or more diuretic or natriuretic molecules within a specified range using feedback obtained from sensors configured to detect information from a patient. The devices, systems and methods can administer one or more diuretic or natriuretic molecules subcutaneously, intramuscularly, intradermally or intravenously. The devices, systems and methods of the invention can deliver any one or a combination of the Atrial Natriuretic Peptide (ANP) hormones. A non-exhaustive includes long-acting natriuretic peptide (LANP), kaliuretic peptide (KP), urodilatin (URO), atrial natriuretic peptide (ANP), brain natriuretic peptide (BNP) and vessel dilator (VD). Other peptides can be delivered including chimeric natriuretic peptides, such as but not limited to CD-NP, CU-NP, or BD-NP

### DEFINITIONS

The term “natriuretic peptide” refers to any peptide, polypeptide or oligopeptide that can increase natriuresis or diuresis in the body. In particular, a natriuretic peptide is a peptide that binds to a natriuretic peptide receptor to regulate the activity of guanylyl cyclases.

The term “sensor” refers to any device or apparatus that can measure a parameter associated with the physiological state of a patient including, but not limited to, any of the parameters listed in Table 1 herein.

The term “on patient's person” or similar terms means that a sensor, processor or other device has an association with the patient such that the sensor, processor or device would relocate with an ambulatory patient.

The term “in the vicinity of the patient” refers to a processor or other device that while not necessarily physically attached to the patient, is within a close physical distance to the patient to have direct wireless or telemetry communication with the patient, such as wireless communication with Bluetooth or 802.11 network protocols.

The term “closed loop” refers to a system or method wherein information obtained from the patient, for example by the use of one or more sensor or through data provided by a clinician, is used to make modifications to a treatment protocol after the protocol has begun.

The term “open loop” refers to a system or method wherein information obtained from the patient, for example by the use of one or more sensor or through data provided by a clinician, is used to establish a treatment protocol designed having a start point and an end point, where further data is not used to modify the treatment protocol between the start point and the end point.

The term “cardiovascular parameters” refers to measurements that indicate the cardiovascular function of the patient. Cardiovascular parameters include, but are not limited to, blood pressure, pulmonary artery pressure, left atrial pressure, right atrial pressure, central venous pressure, and cardiac output.

The term “blood pressure” refers to one or more of the maximum (systolic) and minimum (diastolic) arterial pressure exerted by the heart on the systemic vascular system, particularly, but not limited to, pressure on the brachial artery.

The term “pulmonary artery pressure” refers to the mean pressure found within the pulmonary artery due to action of the heart.

The term “left atrial pressure” refers to the pulmonary capillary wedge pressure measured by wedging a pulmonary catheter with an inflated balloon into a small pulmonary arterial branch, which provides an indirect measurement of pressure in the left atrial chamber of the heart.

The term “right atrial pressure” or “central venous pressure” refers to the pressure in the thoracic vena cava near the right atrium of the heart, which reflects the amount of blood returning to the heart.

The term “cardiac output” refers to the volume of blood being pumped by the heart, by both the left and the right ventricle, in the time interval of one minute.

The term “kidney parameters” refers to measurements that indicate the effectiveness of the kidneys in removing substances and/or fluid from the blood. Kidney parameters include, but are not limited to, proteinuria, plasma renin, and glomerular filtration rate (“GFR”).

The term “proteinuria” refers to a condition in which urine contains an abnormal amount of protein. Albumin is the main protein in the blood; the condition where the urine contains abnormal levels of albumin is referred to as “albuminuria.” Healthy kidneys filter out waste products while retaining necessary proteins such as albumin. Most proteins are too large to pass through the glomeruli into the urine. However, proteins from the blood can leak into the urine when the glomeruli of the kidney are damaged. Hence, proteinuria is one indication of kidney disease (KD).

The term “plasma renin” refers to the amount or activity of renin enzyme found in the plasma of the blood that is involved in regulating arterial pressure.

The term “glomerular filtration rate (GFR)” describes the flow rate of filtered fluid through the kidney. The estimated glomerular filtration rate or “eGFR” is a measure of filtered fluid based on a creatinine test and calculating the eGFR based on the results of the creatinine test. However, other methods are known for estimating GFR and GFR estimation is not limited to the creatinine test.

The term “impedance measurement” refers to the opposition provided by a tissue or anatomical structure to the passage of an electrical alternating current. The SI unit for impedance measurements is ohms.

The term “therapeutically effective amount” refers to an amount of an agent (e.g., chimeric diuretic or natriuretic molecules) effective to treat at least one symptom of a disease or disorder in a patient or subject. The “therapeutically effective amount” of the agent for administration may vary based upon the desired activity, the disease state of the patient or subject being treated, the dosage form, method of administration, patient factors such as the patient's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

The terms “treating” and “treatment” refer to the management and care of a patient having a pathology or condition for which administration of one or more therapeutic compounds or peptides is indicated for the purpose of combating or alleviating symptoms and complications of the condition. Treating includes administering one or more formulations or peptides of the present invention to prevent or alleviate the symptoms or complications or to eliminate the disease, condition, or disorder. As used herein, “treatment” or “therapy” refers to both therapeutic treatment and prophylactic or preventative measures. “Treating” or “treatment” does not require complete alleviation of signs or symptoms, does not require a cure, and includes protocols having only a marginal or incomplete effect on a patient or subject.

The term “therapeutic regimen” is used according to its meaning accepted in the art and refers to, for example, a part of a treatment plan for an individual suffering from a pathological condition that specifies factors such as the agent or agents to be administered to the patient or subject, the doses of such agent(s), the schedule and duration of the treatment, etc.

The term “chimeric peptide(s),” as used herein is defined as artificial construct(s) consisting of bioactive compounds from at least two different peptides or two sequences from different parts of the same protein. Such multifunctional peptide combinations are prepared to enhance the biological activity or selectivity of their components. New biological effects can also be achieved with the chimera. In accordance with the present invention, the chimeric peptides are fusion peptide construct comprising different portions of any one of the natriuretic peptides.

The term “diuretics” means a drug that promotes the formation of urine by the kidney. Diuretics cause a person to lose water, by inhibiting the kidney's ability to reabsorb sodium, thus enhancing the loss of sodium and consequently water in the urine (high ceiling loop diuretic); enhancing the excretion of both sodium and chloride in the urine so that water is excreted with them (thiazide diuretic); and blocking the exchange of sodium for potassium, resulting in excretion of sodium and potassium but relatively little loss of potassium (potassium-sparing diuretic). The categories of diuretics include, but are not limited to, high ceiling loop diuretics, thiazides, carbonic anhydrases inhibitors, potassium-sparing diuretics, calcium-sparing diuretics, osmotic diuretics, and low ceiling diuretics. Other examples of diuretics include furosemide, ethacrynic acid, torsemide, bumetanide, hydrochlorothiazide, thiazides, acetazolamide, methazolamide, spironolactone, potassium canreonate, amiloride, triamterene, and glucose. Preferred embodiments of the invention include diuretics that can be delivered intravenously and/or in a clinical setting

### Medical Devices and Methods

The blood volume and fluid regulation of the kidneys is controlled by a feedback system involving the nervous system and hormonal signals. The build-up of congestion during heart failure can be addressed through the administration of diuretic or natriuretic molecules. Diuretic or natriuretic molecules act on the renal tissues to affect the removal of fluid from the body and can thereby reduce blood volume and fluid retention. However, the use of diuretic or natriuretic molecules can also result in a sudden drop in blood volume and blood pressure. As such, the dosing regimen of diuretic or natriuretic molecules needs to be controlled in order to protect the patient from adverse effects.

The medical devices, related systems and methods contain one or more sensors adapted to detect at least one physiologic parameter in a patient relating to any one of cardiac function, kidney function or fluid status. The devices and systems further contain processing and control components to adjust and/or monitor a therapy to a patient based on a determination of the parameters received from the sensors. Control can be provided in either an open loop or closed loop manner. The sensor data can be described as feedback received from a patient based on any one of central venous (“CV”) parameters, kidney parameters, and impedance measurements. The CV parameters can include but are not limited to blood pressure, pulmonary artery pressure, left atrial pressure, right atrial pressure, central venous pressure, and cardiac output. The kidney parameters can include but are not limited to proteinuria, plasma renin, and glomerular filtration rate (“GFR”). Impedance measurements indicative of lung congestion can be obtained by known measurement systems and devices such as the OptiVol Fluid Status Monitoring, commercially available from Medtronic, Inc., Minneapolis, Minn.

The devices, systems and methods administer the one or more diuretic or natriuretic molecules subcutaneously, intramuscularly, or intravenously and monitor and adjust the dose of the drug within a specified range using sensors configured to detect or monitor one or more parameters in the patient. The devices, systems and methods of the invention are useful for treating renal or cardiovascular diseases, such as congestive heart failure (CHF), dyspnea, elevated pulmonary capillary wedge pressure, chronic renal insufficiency, acute renal failure, cardiorenal syndrome, and diabetes mellitus.

In certain embodiments, the medical device is in a closed loop and determines a parameter or substance of interest to adjust, monitor, and deliver a therapeutic dose of one or more or natriuretic molecules to the patient using a pump once treatment has initiated. The system determines an appropriate response based on the determined parameter or substance of interest and instructs the pump accordingly. The control system can be integrally connected to a sensor module, a drug provisioning component that dispenses an appropriate amount of one or more diuretic or natriuretic molecules to the patient, and a telemetry system for communicating information from the control system to an implantable or external drug pump.

One or more sensors may be implanted at a single site in a patient to determine information for a parameter relating to any one of cardiac function, kidney function or fluid status or a substance of interest. Alternatively, a plurality of parameters may be read from a single sensor implanted at the single site in the patient. One method of sensing multiple parameters includes the implantable sensor having a plurality of implantable sensing elements, and reading an output from at least one of the implantable sensing elements. As such, a plurality of parameters may be read from the implantable sensor at the single site. The output from at least one of the implantable sensing elements may be a quantifiable value. At least one of the implantable sensing elements may be a biological parameter sensor, a physiological parameter sensor or an analyte sensor. The sensors of the present invention can optionally be inserted into the vasculature of the patient. The implantable sensing elements can include those that respond to blood pressure, systolic blood pressure, potassium or pH. According to other embodiments of the present invention, sensors may be positioned in the peritoneal space or may be positioned subcutaneously depending on the parameter or substance to be measured.

The invention can automatically adjust the therapy wherein the devices, systems and methods develop an optimal dosing regimen to provide closed-loop therapy based on feedback from the sensors. For example, after the medical device or other component of the system provides information to the computer system containing the algorithm, an algorithm reviews and adjusts the dosing as necessary. The devices, systems and methods may store a table or other data structure that contains records, in which each record contains dosing data associated with a respective value of a patient parameter. The devices, systems and methods automatically update the table in response to feedback from sensors in the patient, or update the table after receiving confirmation that an adjusted dose is desired. The devices, systems and methods can update the program table after feedback measurement from the patient, after completing a dosing therapy that includes a number of inputs, or periodically during dosing therapy.

Measured parameters include but are not limited to blood pressure, central venous pressure, diastolic and systolic pressures, pulmonary artery pressure, change in cardiac pulse pressure, heart rate measures (ECG), creatinine, or any other parameters indicative of fluid status in a patient. For example, hypervolemia can be measured by pulmonary arterial pressure, renal arterial pressure, and peripheral edema.

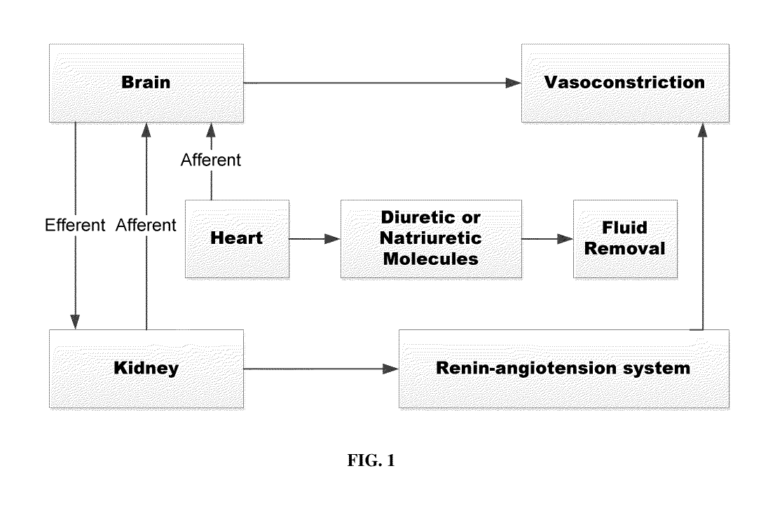

FIG. 1 shows a modified schematic for the regulation of water retention, blood volume and cardiovascular constriction including a step for administration of diuretic or natriuretic molecules. As shown, the renal nerve from the kidneys and the cardiovascular system send afferent signals to the brain and central nervous system that result in vasoconstriction to increase blood pressure. Consequently, efferent nerve signals from the central nervous system to the kidney stimulate the release of renin, which is an enzyme that activates the renin-angiotension system and further affects blood pressure through vasoconstriction and fluid retention. The vasoconstrictive mechanisms and the renin-angiotension system are often activated by decrease in cardiac output as well as by loss of renal function in kidney disease. Additionally, as blood volume and blood pressure increases, stretching of the wall of the atrium can stimulate the release of atrial that can act on the kidneys to trigger fluid release and suppression of the pathways that stimulate blood pressure increase and fluid retention. As such, heart failure and/or kidney failure patients can be treated by the administration of diuretic or natriuretic molecules either through introduction of a pharmaceutical composition or by stimulating release of endogenous diuretic or natriuretic molecules as shown in a step between the heart and fluid removal. However, there is an overlap in the body pathways that signal for the excretion of fluids and other physiological responses such as vasorelaxation. As such, treatment of heart failure and/or kidney failure patients can result in inadvertent hypotension as well as other undesirable effects.

The methods of the invention contains steps wherein a sensor signal is received that varies as a function of a parameter associated with fluid status, cardiac function or kidney function of a patient. The sensors detect the cardiac, kidney or fluid status event based on the sensor signal and monitor or adjust the therapy. For example, the device monitors parameters to diagnose the condition of the patient subsequent to an initial time period of therapy. The sensors collect data wherein the information is reviewed by a program to make adjustments. In some cases, the program may tailor in real-time a therapy according to the data received from sensors implanted within the patient.

The therapy systems and methods can stop delivery of therapy based on changing therapy parameters upon determining the patient has successfully reached a specified fluid status or blood pressure. In some configurations, a first determination that the patient is in a specified stated based on sensor feedback can be confirmed by a second determination based on another source of sensor feedback.

### Device

The medical device of the present invention includes a subcutaneous device that can be positioned in the patient to be implanted using any non-intravenous location of the patient such as below the muscle layer. The subcutaneous device can also be positioned in the loose connective tissue between the skin and muscle layer of the patient. The electronic circuitry employed in a subcutaneous device can take any form known to those of ordinary skill. It will be understood that conventional components and circuitry such as digital clocks, power supply for powering the circuits and providing telemetry circuits for telemetry transmissions between the device and external programmer are contemplated by the invention. The subcutaneous device function can be controlled by means of software, firmware and hardware that cooperatively monitor the dosing regimen and determine when to deliver, increase, decrease or stop delivery of a drug. The device can also monitor and adjust the dose rate as required.

The medical devices and systems include a drive mechanism contained in the housing operatively coupled to the reservoir to deliver the fluid from the reservoir into the patient's body. The infusion device further includes a processor contained in the housing, and a memory coupled to the processor adapted to monitor a predetermined blood pressure threshold. The sensors of the invention are coupled to the processor and adapted to provide an output signal as a function of any one of fluid status, cardiac function or kidney function. The processor is adapted to compare the sensor output signal with the predetermined blood pressure threshold, and to control the infusion device based on the comparison.

The medical devices and systems of the present invention further include a memory coupled to a processor. For example, the memory can be adapted to store a predetermined blood pressure threshold for a particular patient. If the blood pressure output signal from a sensor exceeds the predetermined blood pressure threshold, the processor causes the indicator to provide an alarm or a warning. Alternatively, the processor is adapted to control the infusion device by causing the drive mechanism to alter delivery of the fluid into the patient's body. In further alternative embodiments, the infusion device also includes a transmitter/receiver coupled to the processor and adapted to communicate with a remote device. The processor is adapted to control the infusion device by causing the transmitter/receiver to send information to the remote device. In certain embodiments, the infusion device further includes an indicator operatively coupled to the processor and adapted to provide information to the patient, computing center, physician or clinician about the blood pressure signal. In response to detected parameters, the medical device and system may alter operation of the pump, provide alarm or text messages, and/or transmit data about the detected conditions to another device or system.

Examples of external pump type delivery devices are described in U.S. patent application Ser. No. 11/211,095, entitled “Infusion Device And Method With Disposable Portion” and Published PCT Application No. WO 01/70307 (PCT/US01/09139), entitled “Exchangeable Electronic Cards For Infusion Devices”, Published PCT Application No. WO 04/030716 (PCT/US2003/028769), titled “Components And Methods For Patient Infusion Device,” Published PCT Application No. WO 04/030717 (PCT/US2003/029019), titled “Dispenser Components And Methods For Infusion Device,” U.S. Patent Application Publication No. 2005/0065760, titled “Method For Advising Patients Concerning Doses Of Insulin,” and U.S. Pat. No. 6,589,229 titled “Wearable Self-Contained Drug Infusion Device,” each of which is incorporated herein by reference in its entirety. Programmable controls operate the drive motor continuously or at periodic intervals to obtain a closely controlled and accurate delivery of the medication over an extended period of time. Such infusion pumps administer one or more diuretic or natriuretic molecules with exemplary pump constructions and systems being shown and described in U.S. Pat. Nos. 4,562,751; 4,678,408; 4,685,903; 5,080,653; 5,097,122; 6,248,093; 6,362,591; 6,554,798; and 6,555,986, which are incorporated by reference herein.

### Telemetry

Examples of communication between the medical device and systems of the present invention and a remote device or system via a remote data communication network are described in U.S. application Ser. No. 11/414,160, entitled “Remote Monitoring for Networked Fluid Infusion Systems,” which is herein incorporated by reference. For example, the pump may transmit information based on data from the sensors to a remote device carried by a physician via a computer network, pager network, cellular telecommunication network, satellite communication network. Additionally, the memory can be adapted to store values associated with the outputs of the sensors associated with predetermined blood pressure, fluid status and kidney status levels. For example, a programmer can be in telemetric communication with subcutaneous device by an RF communication link. The communication link can be any appropriate RF link such as Bluetooth, WiFi, MICS, or as described in U.S. Pat. No. 5,683,432 “Adaptive Performance-Optimizing Communication System for Communicating with an Implantable Medical Device” incorporated herein by reference in its entirety.

In certain embodiments, the invention includes a telemetry circuit that enables programming by means of external programmer via a 2-way telemetry link. Uplink telemetry allows device status and diagnostic/event data to be sent to external programmer for review. Downlink telemetry allows the external programmer to allow the programming of device function and the optimization of the detection and therapy for a specific patient. Known programmers and telemetry systems suitable for use in the practice of the present invention are contemplated by the invention. Programmers typically communicate with an implanted device via a bi-directional radio-frequency telemetry link, so that the programmer can transmit control commands and operational parameter values to be received by the implanted device, so that the implanted device can communicate diagnostic and operational data to the programmer. Programmers suitable for the purposes of practicing the present invention include the Models 9790 and CareLink programmers, commercially available from Medtronic, Inc., Minneapolis, Minn.

Various telemetry systems for providing the necessary communications channels between an external programming unit and an implanted device have been developed and are well known in the art. Telemetry systems suitable for the present invention include U.S. Pat. No. 5,127,404, entitled “Telemetry Format for Implanted Medical Device”; U.S. Pat. No. 4,374,382, entitled “Marker Channel Telemetry System for a Medical Device”; and U.S. Pat. No. 4,556,063 entitled “Telemetry System for a Medical Device.”

### Controller

The controller calculates and issues commands that affect the rate and/or frequency that a pump delivers one or more diuretic or natriuretic molecules. The controller may take the form of an external device or an implantable device. The controller may be programmed for either automatic or manual operation. Upon detection of a cardiac function, kidney function or fluid status irregularity, the controller may automatically start or end dosing, or increase or decrease the rate of dosing.

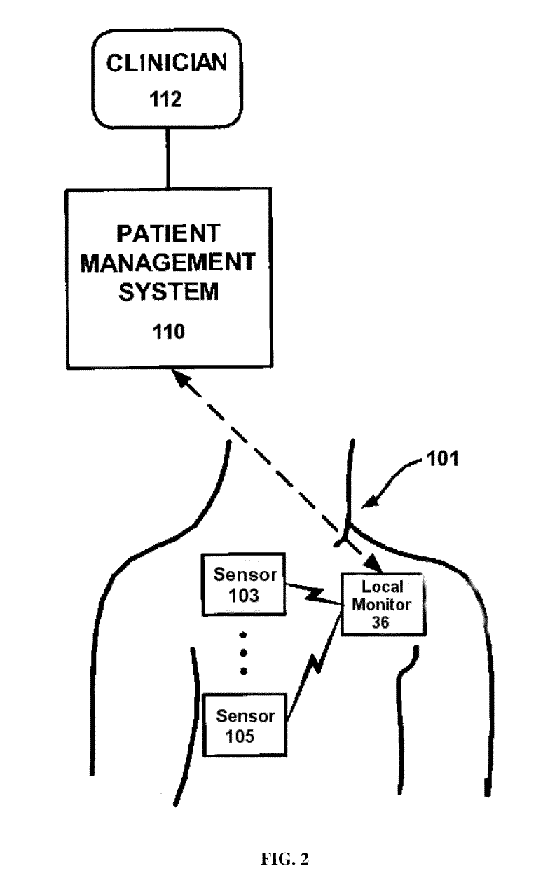

FIG. 2 shows a schematic for a system in accordance with some embodiments. A primary sensor 103 and optionally one or more secondary sensors 105 collect data regarding one or more physiological parameters from a patient 101. The data from the sensor 103 and/or 105 can be optionally processed by a processor or local monitor 36 located on the patient's person or within the vicinity of the patient. The sensor 103 and/or 105 can be implanted within the patient, for example, implantable cardiac/ECG monitors are known as well as the OptiVol® system. Alternatively, the sensor 103 and/or 105 may be external to the patient and optionally on the patient's person or in physical contact with the patient as is common for automatic blood pressure monitoring systems. The sensor 103 and 105 can be present on the patient along with the processor or implanted in the patient in a manner such that telemetry data from the sensors 103 and 105 can be wirelessly transmitted to a patient management system 110 which may be monitored by a clinician or other medical professional 112. It is understood that the patient management system 110 is optional and that the local monitor 36, when present, can act autonomously to collect information from the patient and make control decisions regarding the delivery of diuretic or natriuretic molecules to the patient 101. For example, the local monitor 36 or patient management system 110 can compute an infusion rate that is transmitted to a drug provisioning component 115 having a reservoir containing a composition with one or more diuretic or natriuretic molecules. As such, the diuretic or natriuretic molecules are delivered to the patient 101.

In some embodiments, the primary sensor 103 can be a device that monitors the extent of cardiac function, kidney function or fluid status. In particular, the fluid status can indicate the extent of fluid build-up in the lungs through an intrathoracic impedance measurement. In some embodiments, the primary sensor 103 measures the extent of the patient's need for treatment with diuretic or natriuretic molecules to increase fluid removal/or and to improve kidney function. For example, the primary sensor 103 can be the OptiVol® (Medtronic, Inc.) fluid monitoring system. In addition to fluid monitoring, the OptiVol® system can also provide an indication of pulmonary capillary wedge pressure and pulmonary artery diastolic pressure through intrathoracic impedance measurement. The primary sensor data can be supplemented with additional data that provides an indication of kidney function such as proteinuria or GFR. Kidney function parameters such as proteinuria and GFR can be determined through standard laboratory tests and inputted by a patient, user or physician into the local monitor 36 or patient management system 110. In some embodiments, the primary sensor 103 can be an automated urine volume or output sensor that can evaluate the extent of kidney function. In additional embodiments, the primary sensor 103 can be a device that can automatically determine urea or creatinine concentrations and provide an estimate of GFR. Such automated methods or systems are known in the art (Wei et al. “Fullerene-cryptand coated piezoelectric crystal urea sensor based on urease,” Analytica Chimica Acta, 2001, vol. 437, pp 77-85). In general, data obtained from the primary sensor 103 is used to evaluate the need for treatment with diuretic or natriuretic molecules due to lung congestion or decreased kidney function parameters.

The optional secondary sensor 105 can be an automated blood pressure measuring device for measuring systolic and diastolic pressure, a pulmonary artery pressure monitor, a left atrial pressure monitor or a central venous pressure monitor. The secondary sensor 105 serves to monitor for hypotension or other unfavorable cardiovascular state. Real-time feedback from the secondary sensor 105 can be used to modify the administration regimen of diuretic or natriuretic molecules before or after the initial start of administration to ensure the optimal administration of the peptide. In certain embodiments, the primary sensor 103 serves to collect information regarding blood pressure, pulmonary artery pressure, left atrial pressure or central venous pressure.

TABLE 1
Physiological parameters for measurement
by primary or secondary sensor

| | | Stability of |
|---|---|---|
| Parameter | Indication | Cardiovascular State |
| CV Parameters | | |
| Blood pressure (BP) | | Indicative |
| Pulmonary artery pressure (PA) | Fluid status | |
| Left atrial pressure (LA) | | |
| Right atrial pressure (PA) | Fluid status | |
| Central venous pressure | | Indicative |
| Cardiac output | Cardiac | |
| ECG | Cardiac | Indicative |
| Kidney Parameters | | |
| Proteinuria | Kidney function | |
| Plasma renin | Kidney function | |
| GRF | Kidney function | |
| Urine flow | Kidney function | |
| Creatinine | Kidney function | |
| Urine urea content | Kidney function | |
| Lung congestion | | |
| Optivol ® impedance | Fluid Status | |
| pulmonary capillary wedge | Fluid Status | |
| pressure | | |
| pulmonary artery diastolic | Fluid Status | |
| pressure | | |
| Other | | |
| Peripheral edema | Fluid Status | |
| Splanchnic bed volume | Fluid Status | |
| Serum potassium | Cardiac | Indicative |

Those skilled in the art will appreciate that any one of a wide variety of measurable physiological parameters may be monitored and used to implement the closed-loop adaptive controller described herein. An exemplary controller, used in a closed-loop feedback control for the treatment of peripheral vascular disease, is described in U.S. Pat. No. 6,058,331, the contents of which are incorporated herein by reference. Any one or more of the sensing devices, and/or other physiological sensors, may be employed without departing from the scope of the present invention.

In particular, one method for detecting pulmonary congestion and the fluid status of a patient is to measure intrathoracic impedance. An electrical current is passed across the region of the lung. Since fluid accumulated in the lungs is a better conductor than air, impedance to an applied current will decrease as pulmonary congestion and fluid develops. Alternatively, impedance can be measured non-invasively with surface electrodes, as well as intrathoracically. As such, intrathoracic impedance can optionally provide an indication of the fluid status of a patient and cardiac function. For example, intrathoracic impedance measurements are obtained using implantable devices that are suitable for cardioverter defibrillation or cardiac resynchronization therapy including conventional pacemaker devices. A cardioverter-defibrillator lead is implanted on the right ventricular apex of the heart such that impedance can be measured between the implanted cardioverter-defibrillator lead and the case of the measurement device located in the left pectoral region. A minute ventilation sensor to respond to changes in breathing rate and an accelerometer to respond to physical motion of the patient can also be used. A current is then passed from the device to the right ventricular apex at a frequency asynchronous with the cardiac cycle and the impedance measured. Typically, multiple impedance measurements are collected over a short time of a few minutes and averaged with several hours separating different impedance collection periods. Collecting data over a several minute time span allows averaging to eliminate the effects of cardiac and respiratory cycles. A trend of decreasing impedance can be interpreted by a processor as a sign of pulmonary congestion and deterioration of the fluid status of a patient. Various systems for analyzing impedance data to determine fluid status of the patient and/or a fluid index are known, for example, the OptiVol® Fluid Status Monitoring system (Medtronic, Inc.).

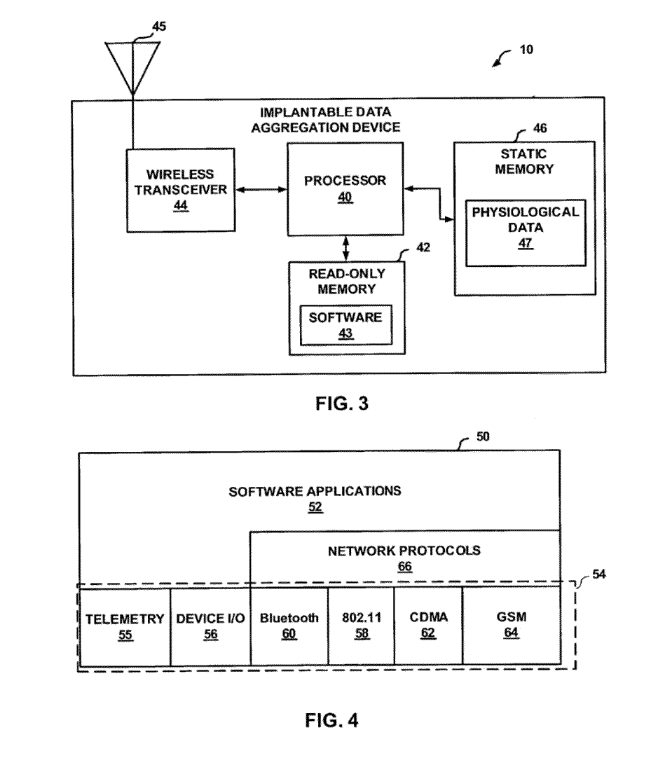

In certain embodiments, data obtained from the one or more sensors is aggregated in a central data aggregation device (DAD) 10, which may be associated with processor 40 of FIG. 3 or the patient management system 110 of FIG. 2. As shown in FIG. 3, the DAD 10 can have an antenna 45 and a wireless transceiver 44. A processor 40 (which can be co-located with patient management system 110) is in communication with read-only memory 42, flash memory (not shown), EPROM (not shown) or other similar memory device containing software 43 for the DAD 10. A non-volatile static memory 46 such as a flash drive or a magnetic media can be provided to serve as a storage area for physiological parameter data 47. The processor 40 can receive signals from the sensors, and process those signals into a form, such as a digital format, which may be analyzed and/or stored in memory 42, such as a dynamic random access memory (DRAM). The memory 42 may also store software, which is used to control the operation of the processor 40.

In one embodiment, signals stored in memory 42 may be transferred via a transmitter (not shown), such as a telemetry circuit, to an external device, such as a programmer. These signals may be stored in the external device, or transferred via a network to a remote system (not shown), which may be a repository or some other remote database. Networks useful with the system of the invention include without limitation, an intranet, internet system such as the world-wide web, or any other type of communication link.

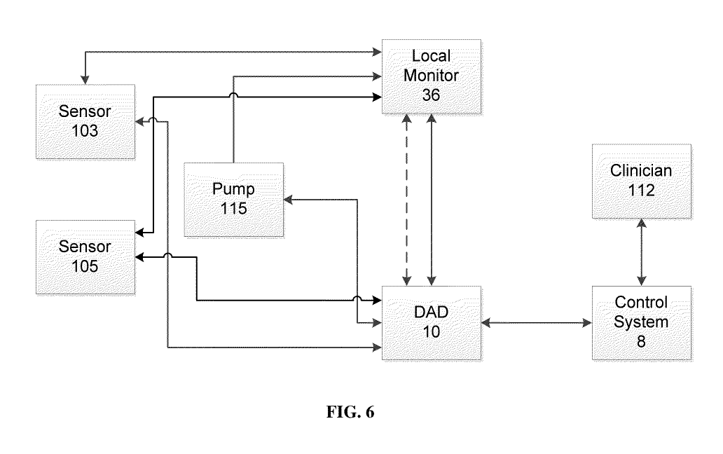

Those skilled in the art will readily understand that the DAD 10 is not required to be a standalone device. The DAD 10 can be incorporated in the same housing as the one or more sensors 103 or the pump 115 (shown in FIG. 6) or can be provided in a separate housing on the patient's person, for example, as part of processor 40 shown in FIG. 3. In other embodiments, the DAD 10 can be present in or as part of a patient management system 110. In certain embodiments, the DAD 10 is located in physical proximity to the pump 115 (FIG. 6) and one or more sensors 103 or 105 (FIGS. 2 and 6) such that data can be transferred using local wireless technology such as Bluetooth or 802.11 rather than receiving data remotely via the internet or via cellular networks.

Those skilled in the art will readily understand that the DAD 10 can store physiological data from the patient. The data stored in the DAD 10 can be accessed by a control system that issues instructions to a pump 115 of FIG. 6 located on the patient's person or implanted in the patient. Due to the functionality of the DAD 10, the control system can be located in any physical local location provided that data from the DAD 10 can be accessed. For example, the control system can be located in a central facility where the data from one or more patients is monitored. Data can be transferred from the DAD 10 to a control system through the use of the internet, the cellular network, cell phone or a landline phone network. Alternatively, the control system can be collocated with a processor on the patient's person or in a patient management system 110 in the patient's vicinity. Moreover, the control system can analyze the physiological data from the patient and makes a command decision regarding the operation of the pump for delivering diuretic or natriuretic molecules to the patient. Operation instructions regarding starting diuretic or natriuretic molecule delivery, stopping diuretic or natriuretic molecule delivery or changing a delivery rate of diuretic or natriuretic molecules can be performed through a direct wireless communication to the pump or delivery through the DAD 10.

FIG. 4 shows a block diagram illustrating an exemplary architecture for the DAD 10. In general, the architecture depicts a number of modules for execution by the processor 40 of FIG. 3 of the DAD 10. The modules can include one or more high-level routines that carryout functions described herein. For example, routines can communicate with one or more sensors (the primary and/or secondary sensor) to collect and aggregate physiological data obtained from a patient. A hardware controller can control a pump 115 of FIG. 6 for delivering the diuretic or natriuretic molecules to the patient. Hardware components can communicate to submit data or obtain commands such as the various sensors and the remote server. Hardware components can make use of one or more communication components, such as GSM, CDMA, 802.11, and Bluetooth. The communication components make use of corresponding chipsets and other hardware components incorporated within hardware components of the system.

For example, the DAD 10 can include a Device I/O driver 56 that can provide an interface to processor-controlled hardware, such as a drug delivery pump 115 of FIG. 6. A telemetry driver 55 can provide an interface for communicating via protocols, such as conventional RF telemetry protocols. A WMTS driver can provide an interface for communication via protocols, such as conventional RF ranges allocated by Federal Communications Commission (FCC) for Wireless Medical Telemetry Service (WMTS). An 802.11 driver 58 can support an 802.11 wireless communication protocol, such as 802.11a, 802.11b, or 802.11g. Similarly, Bluetooth driver 60 can support RF communications according to the Bluetooth protocol. The medical device system can also include CDMA 62 and GSM drivers 64 for supporting cellular communications according to the code division multiple access (CDMA) protocol, or the Global System for Mobile Communications (GSM) protocol, respectively. Software Applications can invoke Network Protocols to make use of these drivers for communication with the pump or a remote server. Network Protocols 66 can implement a TCP/IP network stack, for example, to support the Internet Protocol or other communication protocols. Other protocols may readily be incorporated within the DAD 10.

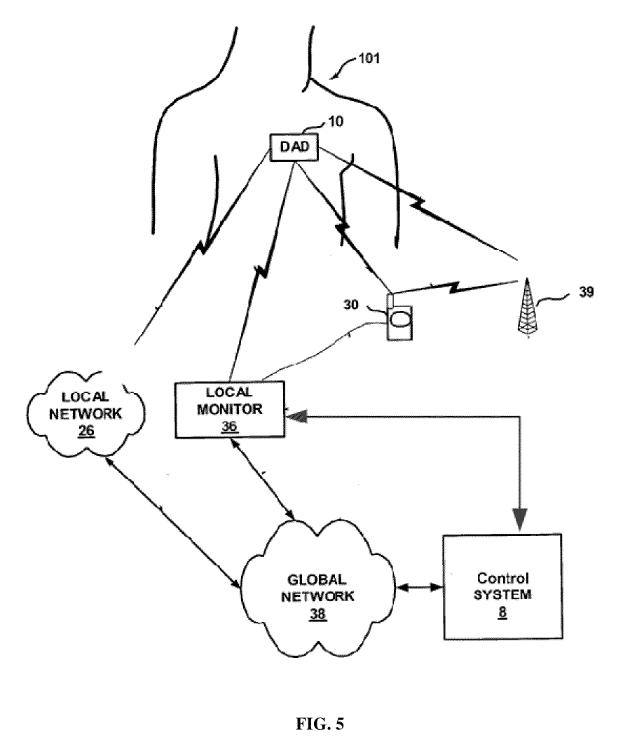

FIG. 5 shows how the DAD 10, regardless of physical location, can serve as a center for communicating information regarding physiological parameter data collected from the patient. As described in FIG. 2, the DAD 10 can function to obtain data from one or more sensors 103 or 105. Such data aggregated by the DAD 10 can be accessed through one or more means as shown in FIG. 5 or the data can be accessed locally to make a control decision regarding delivery of a diuretic or natriuretic molecules to the patient. The DAD 10 can communicate with a local monitor 36, which can optionally be present to allow the patient or another individual in the patient's vicinity to access data from the DAD 10 or input data to the DAD 10. The DAD 10 can be accessed by a local network 26 or a global network 38 (e.g. the Internet) to transfer data to the control system 8. Alternatively, the DAD 10 may communicate directly with the control system 8 through any of the interfaces or means described in FIG. 4. In another embodiment, a cellular phone or other cellular device 30 in the vicinity of the patient can communicate with the DAD 10 through Bluetooth, 802.11 protocols or other suitable wireless means. The cellular phone 30 can then transmit data to any location via the cellular network 39.

The control system 8 of FIG. 5 can contain a processor that makes decisions regarding the administration of diuretic or natriuretic molecules to the patient 101. The control system 8 can be integrated into the patient management system 110 of FIG. 2. Alternatively, the functionality of the control system 8 can be provided at any convenient location provided that the DAD 10 serves to transmit data between the one or more sensors 103 or 105 of FIG. 2, the pump 115 of FIG. 6 and the control system 8 of either FIG. 2 or 5.

As shown in FIG. 6, the DAD 10 maintains communication with the one or more sensors 103/105 and the pump 115. The communication can be direct through a local means of communication (e.g. Bluetooth or 802.11) shown in solid lines or the communication can be through a non-local means (e.g. the Internet, cellular or phone network) shown in dashed lines to a local monitor 36, cell phone 30 of FIG. 5, or similar device. The local monitor 36, cell phone 30 of FIG. 5 or other device is in local communication with the sensors 103/105 and the pump 115 of FIG. 6 The DAD 10 can share any and all needed data with the control system 8 using any of the communication means described in FIG. 4. In some embodiments, the DAD 10 and the control system 8 can be co-located or share the same processor or housing where no distance communication means between the DAD 10 and control system 8 are necessary. Through the control system 8, a clinician or other medical professional 112 can monitor data from the patient, the status of diuretic or natriuretic molecules delivery, etc. and if necessary input data into the system for use by the control system 8 in making a control decision regarding the administration of diuretic or natriuretic molecules.

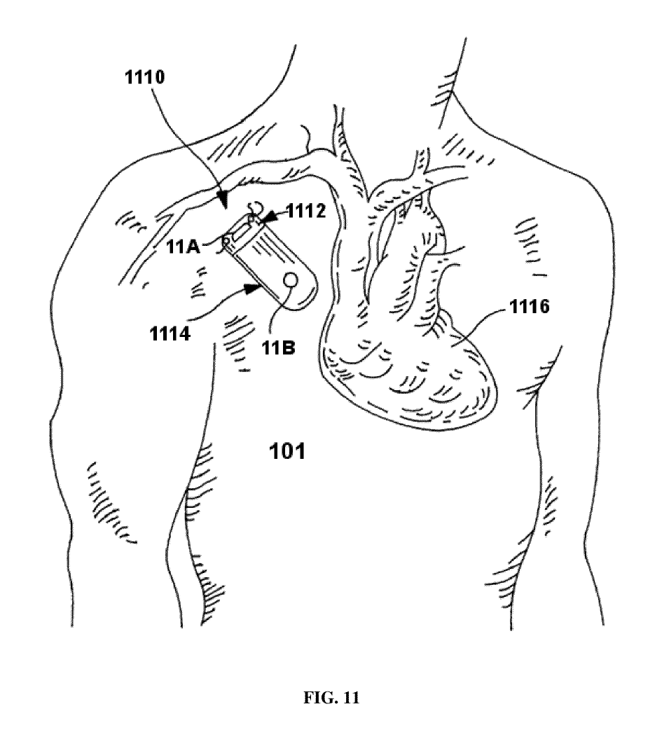

In one embodiment, the local monitor 36 (as shown in FIGS. 2, 5 and 6) can be in communication with the sensor 103 or 105 in some embodiments of the invention, or with the implantable sensor 1110 of FIG. 11 (described in U.S. patent application Ser. No. 13/245,553, entitled “Implantable Monitor,” which is incorporated herein by reference in its entirety). In certain embodiments, the local monitor 36 (as shown in FIGS. 2, 5 and 6) can continuously monitor the heart rhythms of patient 101 using the implantable sensor 1110 of FIG. 11 over an extended period of time, and send erratic heart rhythm data to the control system 8 (as shown in FIG. 5 or 6) that can adjust drug delivery through pump 115 of FIG. 6. The implantable sensor 1110 of FIG. 11 can communicate with control system 8 (as shown in FIG. 5 or 6) through any of the direct wireless or telemetry communication means of FIGS. 2 and 5.

In particular, FIG. 11 illustrates embodiment details of an implantable Cardiac Monitor 1114 implanted subcutaneously in the upper thoracic region of patient 101 and displaced from the patient's heart 1116. The housing 1114 of the monitor 1110 comprises a non-conductive header module 1112 attached to a hermetically sealed enclosure 1114. The enclosure 1114 contains the operating system of the monitor 1110 and is preferably conductive but may be covered in part by an electrically insulating coating. A first subcutaneous sensing electrode 11A is formed on the surface of the header module 1112 and a second subcutaneous sensing electrode 11B is formed by an exposed portion of the enclosure 1114. A feed through extends through the mating surfaces of the header module 1112 and the enclosure 1114 to electrically connect the first sensing electrode 11A with sensing circuitry within the enclosure 1114. The conductive housing electrode 11B can be directly connected to the sensing circuitry within the enclosure 1114.

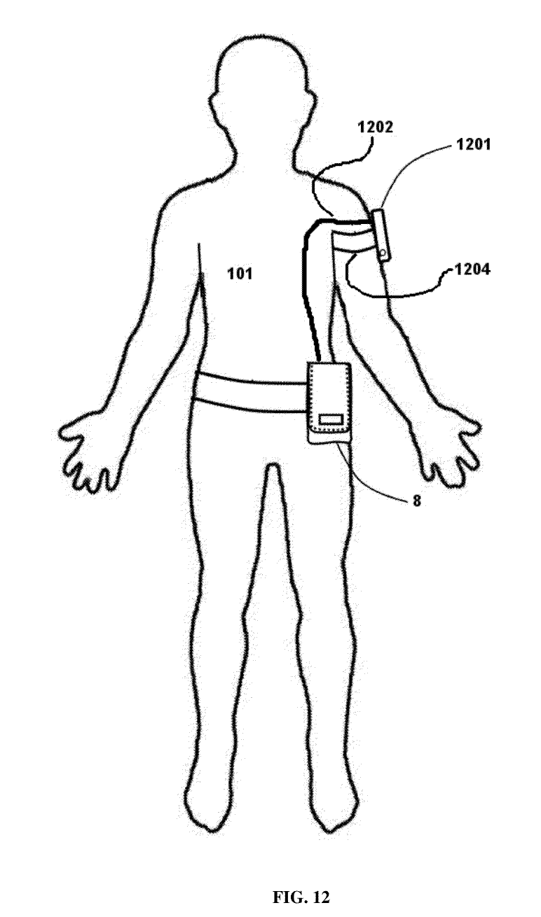

Another embodiment of a sensor 103 can include a wired external blood pressure monitor 1201 as shown in FIG. 12 and described in Blood Pressure Monitoring, October, 2000, Volume 5, Number 4, pp: 227-231, and entitled “Validation of A&D UA-767 device for the self-measurement of blood pressure,” which is incorporated herein by reference in its entirety. In this embodiment, the external local blood pressure monitor 1201, which is held by arm cuff 1204, can monitor the blood pressure and heart rate, and communicate with control system 8 of FIG. 5 through a serial communication cable 1202. Real-time communication can be achieved by sending the blood pressure measurement to control system 8. Control system 8 can adjust the drug delivery through pump 115 of FIG. 6 according to the blood pressure and heart rate of patient 101.

Additional sensors 103 can include sensors that periodically monitor the status of the patient. For example, some patient parameters may only be measureable through direct access to the patient's blood; however, a sensor does not have to continually remain in contact with the patient's blood. In some embodiments, a communication system can indicate the need for certain updated patient parameter data, such as creatinine data or other data concerning blood chemistry, wherein the patient or clinician can apply a point of care device that has means to draw blood (e.g. a finger prick) and determine the needed blood chemistry parameters (e.g. creatinine, serum urea, electrolytes, blood oxygen, glucose, etc.).

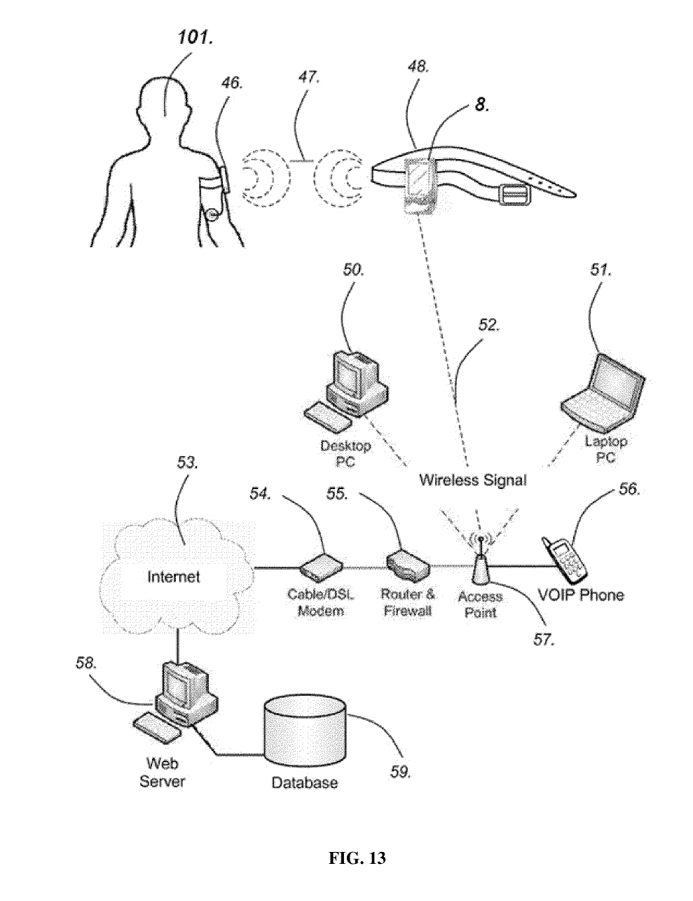

An additional embodiment of a sensor 103 may include as a sensor a wireless external health monitor 46 in FIG. 13. One specific, non-limiting example is the Lifesource Ua-851ant Ehealth Wireless Multi-Function Auto Blood Pressure Monitor. In this embodiment, external health monitor 46 can monitor the blood pressure and heart rate, and communicate with control system 8 through direct wireless or telemetry communication. Control system 8 may adjust the drug delivery through pump 115 of FIG. 6 according to the blood pressure and heart rate of patient 101.

FIG. 13 illustrates the wireless external health monitor 46 integrated with a wireless transmission network and internet based web service for continuous medical monitoring. As depicted, patient 101 is shown wearing wireless monitor 46. Pathological information, in digitized form, and communicated with short range wireless 47, is intercepted with belt 48 worn control system 8, and analyzed to yield information on the patient's blood vital signs. Control system 8 may be programmed to continuously monitor and log the patient's vital signs, and also generate emergency alerts that can be triggered automatically or manually by patient 101. Both blood vital sign data, and alert status, can be communicated to a wireless transmission network through a local access point 57 using a long range broadband wireless signal 52 generated by control system 8. This information can be stored on the patient's desktop PC 50 or laptop PC 51 for the purpose of home monitoring, or communicated to a medical web based internet server 58, offering web services and database storage 59 through the internet 53. Typical internet components such as cable, DSL, or modem connections 54, router 55, or VOIP telecommunication connection 56, may be present in other embodiments.

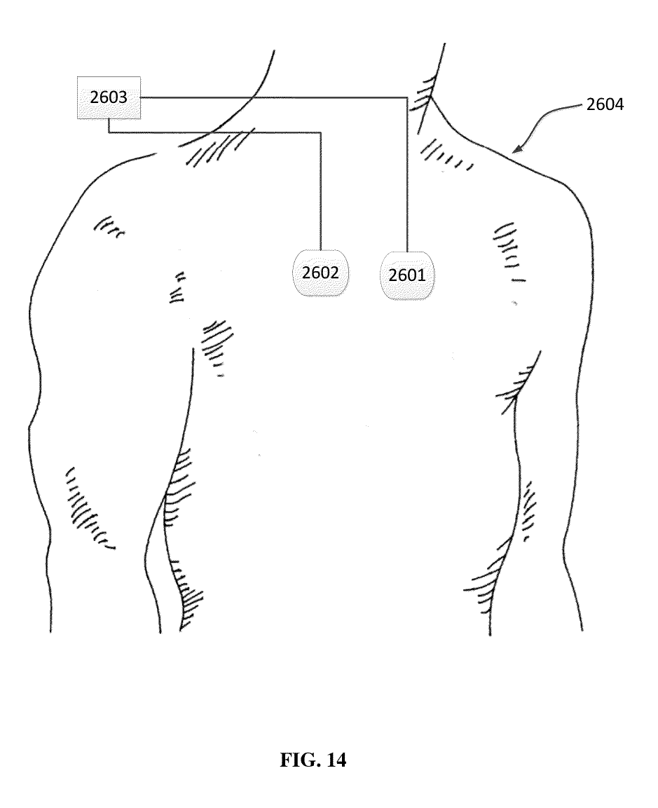

As shown in FIG. 14, certain embodiments of the sensor 103 (described in FIG. 2 or 6) is a non-invasive cardiac output monitoring (NICOM) monitor 2603 which provides a method to measure cardiac data for a living being (described in U.S. Pat. No. 6,676,608, entitled “Method and apparatus for monitoring the cardiovascular condition, particularly the degree of arteriosclerosis in individuals,” which is incorporated herein by reference in its entirety). In one embodiment, the monitor comprises two electrodes (2601, 2602) which carry out two roles simultaneously. The first role is to deliver a continuous low-voltage alternating electrical current. The second role is to sample at a very fast rate the signal emanating from a patient. The signals are then analyzed to determine stroke volume (SV), heart rate (HR), cardiac output (CO), stroke volume variation (SVV) and other hemodynamic information. In one embodiment, the two electrodes 2601 and 2602 are placed on the surface of the chest of a human being 2604 to collect cardiac data. Although a human being 2604 is depicted, one of ordinary skill will understand that any mammal can be the subject. Further, one of ordinary skill in the art will appreciate that the two electrodes 2601 and 2602 may be placed on different locations of the body of the subject to obtain data.

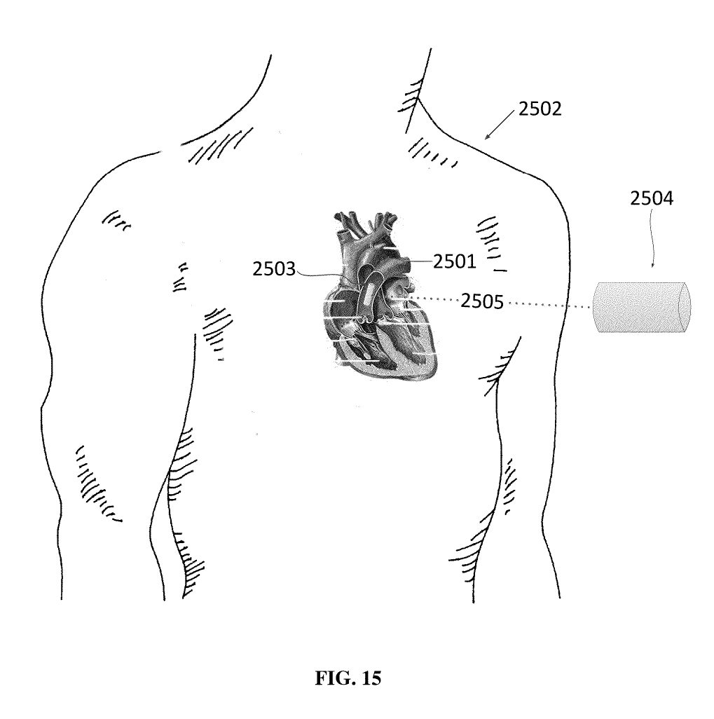

As shown in FIG. 15, an implantable sensor 2503 in another embodiment provides an in-vivo assessment of the fluid pressure and temperature around the sensor 2503 that is positioned in a human being 2502 (described in U.S. patent application publication No. US2012/0016228A1, entitled “System, Apparatus, and Method for In-Vivo Assessment of Relative Position of an Implant,” which is incorporated herein by reference in its entirety). The sensor 2503 comprises a passive electrical resonant circuit that is configured to be selectively electromagnetically coupled to an ex-vivo source of radio frequency (“RF”) energy 2505 and, in response to the electromagnetic coupling, to generate an output resonant signal that is dependent upon the fluid pressure and temperature around the implant monitor at the time of the electromagnetic coupling. An external device 2504 intercepts and analyzes the output signal from the sensor 2503. In one embodiment, the sensor 2503 is self-contained, has no leads to connect to an external circuit and communicates with an ex-vivo monitor by utilizing an inductive-capacitive (“LC”) resonant type circuit. The sensor 2503 can be implanted via catheter on an out-patient basis. In one embodiment, the sensor 2503 is implanted in the pulmonary artery 2501 of human being 2502 to measure the pressure and temperature in the pulmonary artery 2501. Although a human being 2502 is depicted, one of ordinary skill will understand that any mammal can be the subject. One of ordinary skill in the art will also understand that the implantable sensor 2503 can be positioned at different locations within the body to gather desired subject information.

In the embodiments of the invention described in the FIGS. 2 and 5-6, the sensors or monitors can transmit data to a doctor, patient 101 or patient management system 110 in addition to control system 8. Control system 8 can be programmed to adjust the drug delivery automatically or under external permission from the doctor, patient 101 or patient management system 110. As a fail-safe mechanism, control system 8 may be programmed to switch to operate automatically when the blood pressure or hear rate of patient 101 are at a dangerous level. The control system 8 can be provided with various initial criteria to begin an administration regimen. Dosing regiments for diuretic or natriuretic molecules, as with other pharmaceuticals, are often determined through easily available criteria such as weight and severity of symptoms. The control system 8 can be provided with several pieces of information about the patient such as weight, blood pressure, heart rate, biomarkers (BNP, NT-pro BNP, ANP, CNP, NT-CNP, etc) that give an indication of endogenous natriuretic peptide levels, a score of signs of edema and/or a rating symptom severity such as breathing and difficulty with physical activity/walking, and objective measurements of physical condition such as intracardiac pressure, central venous pressure and fluid as measured by intrathoracic impedance. An initial dosing regimen can be arrived at using standard considerations.

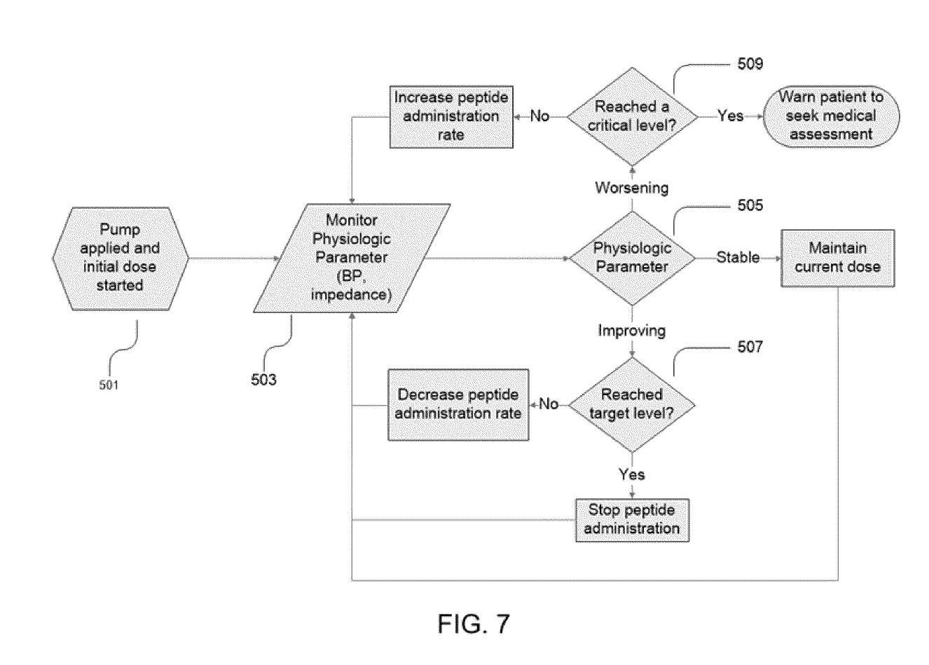

FIG. 7 presents a decision flow chart increasing or decreasing an administration rate of diuretic or natriuretic molecules. In some embodiments, the decision to increase or decrease an administration rate can be made based upon the observance of one physiological parameter. In step 501, a pump is started or is in operation to deliver diuretic or natriuretic molecules at a first rate. In step 503, a physiological parameter from the patient is observed. In some embodiments the physiological parameter indicates the stability of blood pressure levels. In other embodiments, the physiological parameter indicates the severity of symptoms of heart failure or kidney failure, such as a fluid measured by OptiVol® impedance. Where a parameter from Table 1 associated with the stability of the patient is measured, the flow chart of FIG. 7 can adjust the dose to a level that does not cause hypotension. In step 505, a physiological parameter such as blood pressure is compared to a previous measured value of the physiological parameter. If BP is substantially changed compared to a previous measurement, then the decision flow chart advances to step 507. At step 507, if BP has reached a critically low level that indicated hypotension (Decision 1), then the administration of the peptide is stopped. At step 507, if BP has decreased a significant amount but not reached a level that indicates hypotension, then the rate of administration is decreased.

In some embodiments, hypotension is indicated by a systolic blood pressure (SBP) of 110 mmHg or less. In other embodiments, hypotension is indicated by a systolic blood pressure of 100 mmHg or less. In certain embodiments, a significant decrease in blood pressure is indicated by a change of systolic blood pressure of more than about 5 mmHg. In other embodiments, a significant decrease in blood pressure is indicated by a change in systolic blood pressure of more than 7 mmHg.

If BP is not substantially changed compared to a previous measurement, then the decision flow chart advances to step 509 (Decision 2). If the rate of administration has not reached a maximum limit in step 509, then the rate of administration is increased and the physiological parameter is analyzed again in step 503. If the rate of administration has reached a maximum limit in step 509, then the rate of administration is left unchanged and optionally an additional assessment can be performed to determine if the maximum rate of administration for a particular patient should be increased.

In other embodiments, the physiological parameter monitored in step 503 of FIG. 7 provides an indication of renal function, cardiac function or fluid state of the patient. Such physiological parameters, as indicated in Table 1, include GFR, urine urea content, urine flow, pulmonary artery wedge pressure or fluid state as measured by OptiVol® or similar impedance measurement. Improvements in such physiological parameters indicate that the administered natriuretic drug is effective. As used herein, fluid state refers to a state of lung congestion or edema of the patient or any other pathology that indicates an inability of the heart or kidneys to process a sufficient quantity of fluid.

In step 505, a physiological parameter such as fluid state is compared to a previously measured value of the physiological parameter. If the physiological parameter is improved compared to a previous measurement, then the decision flow chart advances to step 507 (Decision 1). At step 507, if the physiological parameter has reached a targeted level for improvement, then the administration of the peptide is stopped and the physiological parameter is further monitored in step 503. If the physiological parameter is improved but has not yet reached a targeted value in step 507, the rate of administration is decreased and the physiological parameter is further monitored in step 503.

In step 505, if the physiological parameter has worsened compared to a previous measurement, then the decision flow chart advances to step 509 (Decision 2). If the rate of administration has reached a maximum rate, then the rate of administration is increased and the system is returned to monitoring the physiological parameter in step 503. If the maximum level of administration has been reached in step 509, then in step 511 the patient is warned to seek additional medical attention. In other embodiments, a hardware component can send a signal that the patient needs medical assistance to a remote location, such as through a telephone or cellular network or the Internet as described herein.

In some embodiments, the amount of increase of the administration rate can be set to be a percentage of the maximum dose. The administration rate is increased by any selected from about 1% to about 10%, from about 2% to about 15%, from about 3% to about 10% and from 2% to about 10% of the maximum dose. In some embodiments, the amount of increase in an administration rate can depend upon the difference of the current administration rate from the maximum rate.

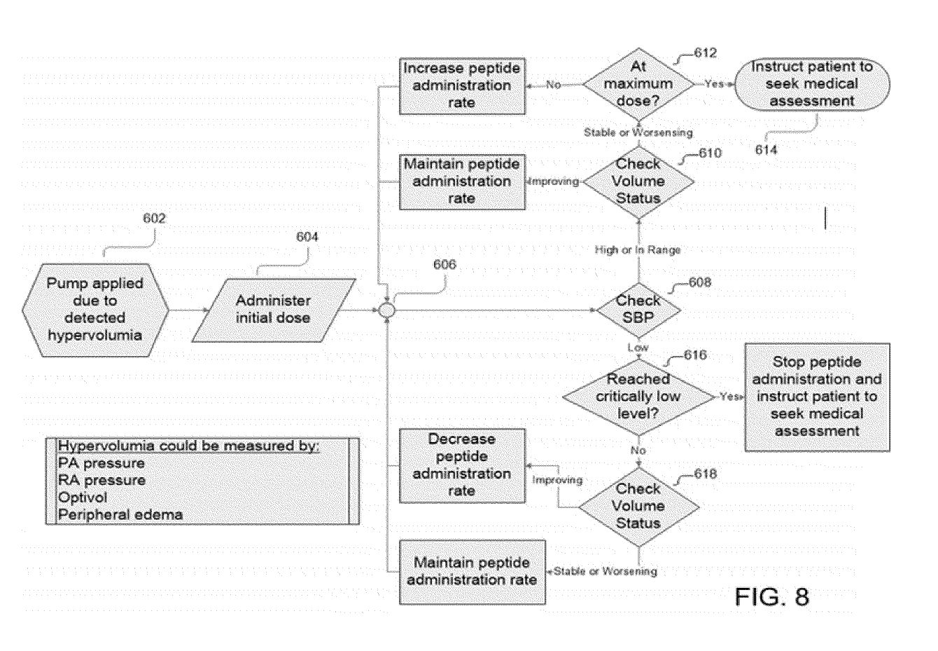

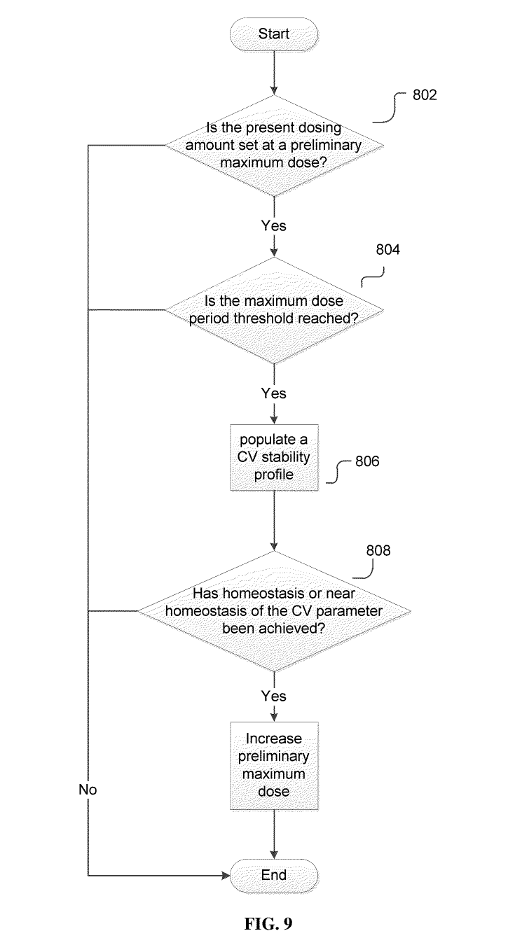

In FIG. 8, a decision flow chart is shown wherein the rate of administration can be adjusted by a control system from monitoring at least two physiological parameters. In step 602, a routine for the detection of hypervolumia or decrease in kidney function is detected and an initial administration rate of diuretic or natriuretic molecules is initiated in step 604. In step 606, two or more physiological parameters are monitored. In one embodiment, one of the two physiological parameters is a cardiovascular parameter that indicates the stability or instability of the patient, such as systolic blood pressure (SBP) or other physiological parameter indicates renal function, cardiac function or fluid state of the patient. In step 608, the first parameter is compared to a prior measurement of the first parameter or is compared to a reference value or range of values. If the measured first parameter is within an acceptable range, then the decision flow chart advances to step 610, where the second physiological parameter is compared to a prior-measured value. If the second physiological parameter indicates an improvement in kidney function, cardiac function or fluid status, then the rate of administration is maintained and the system is returned to step 606. If the second physiological parameter indicates a worsening of kidney function, cardiac function or fluid status, then in step 612 the rate is set at a maximum rate. If a maximum rate of administration has not been reached, then the rate of administration is increased and the system is returned to step 610. If the maximum rate has been reached, then in step 614 the patient is warned to seek additional medical attention. In other embodiments, a hardware component can send a signal that the patient needs medical assistance to a remote location, such as through a telephone or cellular network or the Internet as described herein.

If in step 608, the measured first parameter is below a reference value or range, and then in step 616 the first parameter is evaluated for having reached a critically low level. If the patient has a critically low first parameter such as blood pressure, then the patient can be warned to seek medical attention. If the first parameter is not critically low, then in step 618 the second physiological parameter is compared to a prior-measured value. If the second physiological parameter indicates an improvement in kidney function, cardiac function or fluid status, the rate of administration is decreased and the system is returned to step 606. If the second physiological parameter indicates a worsening of kidney function, cardiac function or fluid status or that the functions are stable, then the rate of administration is maintained and monitoring of the physiological parameter is continued in step 606.

The protocols describe increasing administration of diuretic or natriuretic molecules to obtain improvement in fluid status, cardiac function and/or kidney function without placing the patient in a destabilized state as measured by a cardiovascular parameter. In particular, the increase in fluid removal caused by diuretic or natriuretic delivery can cause hypotension that can destabilize the patient and require medical intervention to re-establish sufficient blood pressure. As such, the control system 8 is initially instructed to block increases in the administration rate beyond a maximum dose. This maximum dose is set at an amount that should not cause hypotension in most patients. Nonetheless, as described in FIGS. 6 and 7 the control system 8 of FIG. 5 is configured to guard against administering at a rate that causes a deterioration of cardiovascular stability even at doses less than the maximum dose in the system.

The maximum tolerated dose, which is the highest dose that does not cause a deterioration of cardiovascular stability, can vary significantly from patient to patient. In some instances, a patient can vary in their maximum tolerance for diuretic or natriuretic molecules over time. Further, a large increase in the administration of diuretic or natriuretic molecules or a high initial dosing of diuretic or natriuretic molecules can result in a rapid decrease in blood pressure while a gradual increase in the administration of diuretic or natriuretic molecules can allow for the body's natural homeostasis mechanisms, as shown in part in FIG. 1, to stabilize blood pressure and allow for tolerance to greater rates of administration of the diuretic or natriuretic molecules (shown as an addition step in FIG. 1).

FIG. 9 shows a protocol wherein the control system 8 of FIG. 5 can adjust an initially programmed maximum tolerated dose. The system can be programmed with both an initial maximum dose or preliminary maximum dose and a safety-limit maximum dose, which will not be exceeded by the system under any circumstance. In step 612 of FIG. 7, if the patient shows no improvement in fluid status, kidney function or cardiac function after the safety-limit maximum dose has been reached, seeking medical advice is the most appropriate course of action. However, if only a preliminary maximum dose has been reached, then in certain embodiments the preliminary maximum dose can be increased until the point that the safety-limit maximum dose is reached.

A protocol for increasing a preliminary maximum dose of the control system 8 is shown in FIG. 9. The protocol of FIG. 9 can be executed if the maximum dose reached in step 612 of FIG. 7 is only a preliminary maximum dose less than a safety-limit maximum dose. In step 802 in FIG. 9, whether the present dosing amount is set at a preliminary maximum dose is evaluated. If the result is no, then the protocol is ended and the rate of administration is increased as shown in FIG. 8. If yes, then the protocol advances to step 804 where the control system 8 evaluates the time period that the maximum does has been administered for, which can be referred to as the maximum dose period threshold. That is, the control system 8 can guard against the increase in the preliminary maximum dose to ensure that the preliminary maximum dose is increased under conditions where the patient has reached homeostasis regarding systolic blood pressure or other cardiovascular parameter indicating the stability of the patient. Either the DAD 10 or the control system 8 can monitor the amount of time that the current dosing rate has been administered, which can be referred to as current dosing time.

The precise value of any preliminary maximum dose is not particularity limited. Rather, FIG. 9 describes a manner in which the preliminary maximum dose can be adjusted based upon the observation of actual patient response. The safety-limit maximum dose can be set at a conservative level to prevent an accidental overdose of the diuretic or natriuretic molecules. In some embodiments, the safety-limit maximum is determined by limits set by governmental regulatory approval for the particular pharmaceutical administered. In some embodiments, the maximum dose period threshold is from about 5 minutes to about 60 minutes. In other embodiments, the maximum dose period threshold is from about 60 minutes to about 120 minutes. In still other embodiments, the maximum dose period threshold is any of from about 30 minutes to 60 minutes, from about 60 minutes to about 75 minutes, from about 75 minutes to about 90 minutes, form about 2 hours to about 4 hours and from about 2 hours to about 12 hours.

In step 804, if the current dosing time is less than the maximum dose period threshold, the protocol of FIG. 9 is ended and the control system 8 increases the administration rate as shown in FIG. 8. If the result is yes in step 804, then the system populates a CV stability profile in step 806. In certain embodiments, the CV stability profile contains at least two parameters: the average CV parameter value (e.g. systolic blood pressure) during the current dosing time and the standard deviation of the CV parameter during the current dosing time. Whether the patient has reached a state of homeostasis or near homeostasis with regards to the CV parameter, in certain embodiments, the standard deviation of the CV parameter is divided by the average of the CV parameter to form a quotient, which can be expressed in percent terms by multiplying by 100. In some embodiments, homeostasis or near homeostasis is indicated by a quotient less than about 15%. In other embodiments, homeostasis or near homeostasis is indicated by a quotient less than about 10%. In still other embodiments, homeostasis or near homeostasis is indicated by a quotient less than about 5%.

In step 805, if the division of the standard deviation by the average CV parameter value indicates homeostasis or near homeostasis of the CV parameter, then the preliminary maximum dose is increased to a new preliminary maximum dose. As shown in FIG. 8, the diuretic or natriuretic molecules administration rate can be increased to the new preliminary maximum dose and physiological parameters can continue being observed in step 606. In step 808 of FIG. 9, if the division of the standard deviation by the average CV parameter value indicates that homeostasis or near homeostasis of the CV parameter has not been achieved, then the preliminary maximum dose is not increased. In some embodiments, the administration rate is maintained and the control system 8 returns to step 606 in FIG. 8. In other embodiments, if a no result is obtained in step 808, then the safety-limit maximum dose is set to be the current preliminary maximum dose to prevent any future increases in the administration rate of the diuretic or natriuretic molecules.

In certain embodiments, the amount that the preliminary maximum dose limit is increased by is not particularly limited. After an increase in the preliminary maximum dose, the preliminary maximum dose may still be raised in the future if the safety-limit maximum dose is reached. As such, an increase in the preliminary maximum dose can be moderate while still allowing for the control system 8 of FIG. 5 to adjust the administration rate in a patient-specific manner. In some embodiments, the amount that a preliminary maximum dose is increased by is a percentage of the safety-limit maximum dose. In some embodiments, the amount of increase of the preliminary maximum dose is any selected from about 1% to about 10%, from about 2% to about 15%, from about 3% to about 10% and from 2% to about 10% of the safety-limit maximum dose. In certain embodiments, the amount that a preliminary maximum dose is increased by upon a yes result in step 608 can depend upon how close the current preliminary maximum dose is to the safety-limit maximum dose. In particular, the control system 8 can be programmed to increase the preliminary maximum dose based upon the difference between the current preliminary maximum dose and the safety-limit maximum dose.

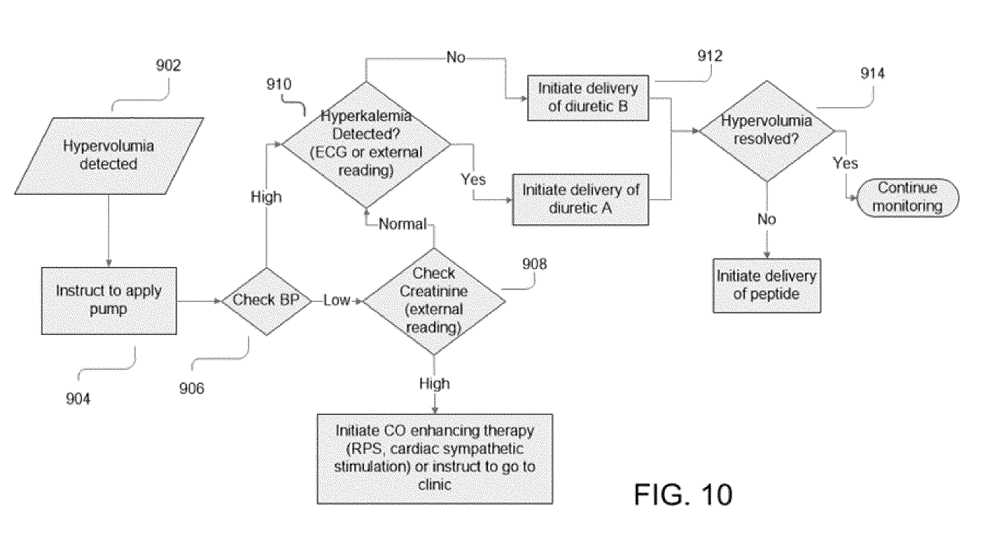

In certain embodiments, the control system 8 can automatically detect hypervolumia or an excess fluid status of the patient to initiate delivery of the diuretic or natriuretic molecules using pump 115 of FIG. 5, the process being shown in FIG. 10. In step 902 of FIG. 10, the control system 8 detects hypervolumia or another HF event, for example, excess fluid build-up can be detected by impedance measurement. In step 904, the pump of the invention is applied. In step 906, the control system 8 checks the patient's blood pressure compared against a threshold. If the patient's blood pressure is below the threshold, then the control system 8 continues to step 908 where the creatinine of the patient is checked. If creatinine is high compared to a threshold, then the control system 8 signals to a clinician or other medical professional to begin a cardiac output (CO) enhancing therapy. If the creatinine level measured in step 908 is within a normal range, then the control system 8 advances to step 910 where the patient is checked for hyperkalemia (blood potassium level). An ECG device or a sensor automatically that receives ECG data can be used to determine if hyperkalemia is present. Alternatively, an external blood test can be performed to identify hyperkalemia.

The presence of hyperkalemia can affect the requirement for a diuretic to be used to remedy the hypervolumia before the administration of diuretic or natriuretic molecules. Specifically, some diuretics more greatly affect the removal of potassium ions from the body than others. If hyperkalemia is detected in step 910, then the control system 8 of FIGS. 2 and 5 signals for the administration of diuretic A, as shown in step 912. If hyperkalemia is not detected in step 910, then the control system 8 of FIGS. 2 and 5 signals for the administration of diuretic B, as shown in step 912. In step 914, the control system 8 of FIGS. 2 and 5 monitors the affect that diuretic treatment has had on hypervolumia using any method appropriate for detecting hypervolumia in step 902. If the hypervolumia state is not resolved, then the control system 8 of FIGS. 2 and 5 initiates the delivery of the diuretic or natriuretic molecules. The delivery of diuretic or natriuretic molecules can be carried out using the protocols described in FIGS. 7 through 8 described herein.

Further shown in FIG. 10, diuretic drugs can be administered prior to administration of a natriuretic peptide to resolve hypervolumia. FIG. 10 shows a procedure for possibly initiating a sequence of treatment with either a diuretic or a natriuretic peptide. If hypervolumia is detected, blood pressure—which can be arterial blood pressure, central venous blood pressure or both—and a marker of kidney stress can be monitored to determine a therapeutic course of action including identifying a proper diuretic to administer.

As shown in step 906, blood pressure, such as arterial blood pressure or central venous blood pressure (“BP”), is determined to be above a critical value (“high BP”) or below a critical value (“low BP”). In one non-limiting case, if systolic blood pressure (SBP) is less than or equal to 90 or diastolic arterial pressure (P_{A}) is less than or equal to 10, then the system can be instructed to stop infusing a therapeutic drug such as VD. If SBP is greater than or equal to 140 or diastolic P_{A} is greater or equal than 22, the system can deliver the therapeutic drug as needed (i.e., as the situation arises wherein the specific dosage is not scheduled but determined by the systems of the invention) or the system can deliver an increased amount of the drug or begin to administer a second drug such as a vasoactive agent.

In each step, the algorithm can check serum creatinine. For example, if BP is deemed to be low, an additional evaluation of at least one marker of kidney stress, as discussed above, is evaluated in step 908. If markers of kidney stress are present, then in some embodiments treatment with a diuretic is not carried out. That is, the system can determine that drug treatment in the presence of markers of kidney stress would carry excess risk where the system can signal that a physician or another clinician should be consulted. Markers of kidney stress can include electrolyte balance and blood urea, which can be determined by automated sensors known to those of ordinary skill in the art. In one embodiment, high serum creatinine level can be determined as a marker of significant kidney stress.

If BP is determined to be high in step 906, then in some embodiments, markers of kidney stress in step 908 need not be evaluated. However, in other embodiments, markers of kidney stress can be evaluated as a further precaution before judging to continue with kidney dialysis. Different criteria can be used in some embodiments to evaluate markers of kidney stress depending upon whether high BP or low BP is observed in step 906.

If markers of kidney stress are not observed in step 908 and/or BP is determined to be high in step 906, then the patient's serum potassium level is determined in step 910. Normal serum potassium level is about 5 mEq/L. Hyperkalemia can be determined automatically by the system by the observations of certain features in an ECG of the patient, which can be continuously monitored. If hyperkalemia is detected, then a diuretic that has properties to remove potassium ions from the body can be selected, which is indicated as diuretic A in FIG. 10. If hyperkalemia is not detected, then a diuretic that results in an attenuated removal of calcium from the body is used, which is indicated as diuretic B in FIG. 10. In some embodiments, diuretic B can be a diuretic administered with a potassium salt and or a mixture of diuretics that includes at least one diuretic that has attenuated potassium removal properties known as potassium sparing diuretics.

In some embodiments, diuretic A can be a diuretic known as a loop diuretic that acts on the Na+-K⁺-2Cl⁻ symporter. Examples of loop diuretics include, but are not limited to, furosemide, bumetanide, etacrynic acid, etozoline, muzolimine, piretanide, tienilic acid and torasemide. Loop diuretics can act to remove potassium ions from the body.

If hyperkalemia is not determined in step 910, then the use of loop diuretic alone can cause undesirable decreases in serum potassium levels. As such, diuretic B is used which has attenuated potassium removal properties compared to diuretic A. In some embodiments, diuretic B is a mixture of more than one diuretic or drug. In some embodiments, diuretic B is a loop diuretic, as described above, administered in combination with a potassium sparing diuretic. Potassium sparing diuretics include epithelial sodium channel blockers such as amiloride and triamterene and aldosterone antagonists such as spironolactone and eplerenone. Further, angiotensin-converting-enzyme (ACE) inhibitors can be used as diuretic B either in combination with a loop diuretic or alone. In other embodiments, diuretic B can be a loop diuretic in administered in combination with potassium. In particular, Lasix-K® is known as a combination of furosemide with potassium. In additional embodiments, diuretic B can be a potassium sparing diuretic and/or an ACE inhibitor without the use of a loop diuretic.

In step 912, either diuretic A or diuretic B is administered using a pump to deliver the drug automatically to the patient. In some embodiments, a pump can accommodate more than one reservoir to accommodate both diuretic A and diuretic B. In other embodiments, diuretic A and diuretic B can be provided in separate reservoirs wherein a communication system can signal to the patient or a health care provider the proper reservoir to be inserted into the device for delivery. In additional embodiments, both the diuretic A and the diuretic B need not be provided, however, the system can continuously verify that an appropriate diuretic is being administered. In step 914, the result of the administered diuretic to resolve the initial hypervolumia is evaluated. If hypervolumia is not resolved, then the system can begin the administration of a natriuretic peptide, wherein methods for the administration of the natriuretic peptide are described herein. If hypervolumia is evaluated to be resolved in step 914, then patient monitoring continues wherein no additional treatment is necessary unless hypervolumia returns.

It will be apparent to one skilled in the art that various combinations and/or modifications and variations can be made for therapeutic regimens depending upon the various physiological parameters observed in the patient. For example, a therapeutic regimen calculated using the systems and methods of the invention may be based on any relevant biological parameter, such as the body weight of a patient. The particular embodiments disclosed above are illustrative only, as the invention may be modified and practiced in different but equivalent manners apparent to those skilled in the art having the benefit of the teachings provided herein. Furthermore, no limitations are intended with respect to the details of construction or the design shown herein, other than as described in the claims below. It is therefore evident that the particular embodiments disclose above may be altered or modified and that all such variations are considered to be within the scope and spirit of the present invention.

All patents and publications referenced herein are hereby incorporated by reference in their entireties. It will be understood that certain of the above-described structures, functions and operations of the above-described preferred embodiments are not necessary to practice the present invention and are included in the description simply for completeness of an exemplary embodiment or embodiments. It is therefore to be understood that within the scope of the appended claims, the invention may be practiced otherwise than as specifically described without departing from the spirit and scope of the present invention.

### Examples of Peptides

The devices, systems and methods can deliver an ANP hormone selected from any one of long-acting peptides (LANP), kaliuretic peptide (KP), urodilatin (URO), atrial natriuretic peptide (ANP), vessel dilator (VD), and chimeric peptides with a feedback mechanism. The invention also contemplates chimeric natriuretic peptides such as CD-NP, which comprises the 22 amino acid human C-type natriuretic peptide (CNP) and the 15 amino acid C-terminus of Dendroaspis natriuretic peptide (DNP) as described in U.S. Pat. No. 7,754,852, and United States Provisional application Ser. No. 13/368,225, the contents of which are incorporated in their entirety by reference. The amino acid sequence for CD-NP is GLSKGCFGLKLDRIGSMSGLGCPSLRDPRPNAPSTSA with a disulfide present between the two cysteines of the sequence. The devices, systems and methods can maintain a specified plasma concentration of the natriuretic or chimeric peptide reached during continuous subcutaneous (SQ) infusion. In each example, feedback obtained from sensors measuring any of the patient's physiological parameters can be used to make a decision to begin, stop, or adjust treatment.

Other chimeric natriuretic peptides are known are contemplated by this invention. For example, CU-NP consists of the 17 amino acid ring of human CNP and the N- and C-termini of urodilatin. The sequence of CU-NP is TAPRSLRRSSCFGLKLDRIGSMSGLGCNSFRY with a disulfide bond between the two cysteines. Further, modified versions of CD-NP are known. One variant of CD-NP is a peptide having the sequence GLSKGCFGRKMDRIGSMSGLGCPSLRDPRPNAPSTSA, which differs in amino acid residues 9-11 compared with CD-NP peptide. Another variant of CD-NP is a peptide having the sequence GLSKGCFGLKLDRISSSSGLGCPSLRDPRPNAPSTSA, which differs in amino acid residues 15-17 compared with CD-NP peptide. The term CD-NP, as used herein, includes variants of CD-NP.

### Pharmacodynamic Study of VD in Canines

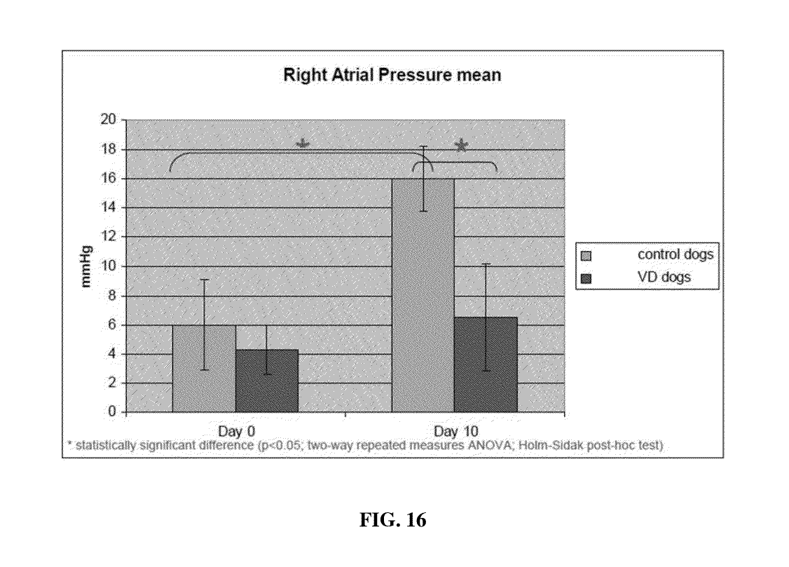

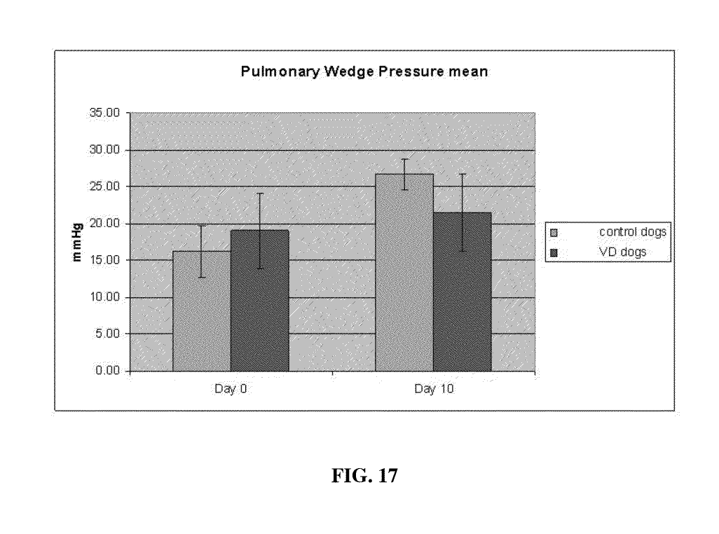

FIGS. 16 and 17 show the mean right atrial pressure and the mean pulmonary capillary wedge pressure, respectively, for control and experimental groups of a canine model high-rate paced (HRP). A pharmaceutical formulation of VD was prepared in a Tris buffer. 16.0 g glycerol, 6.05 g tris-(hydroxymethyl)-aminomethane (“Tris”), 2.50 g meta cresol were mixed in a 1.00 L volumetric flask. Approximately 900 mL nanopure water was added to the volumetric flask and the mixture was magnetically stirred to reach complete dissolution. 4 normal hydrochloric acid was used to adjust pH to 7.3 at 25° C. Then, the flask was filled to 1 L mark with nanopure water. The pH was rechecked and verified to be 7.3 at 25° C. The pH 7.3 Tris buffer was stored at 2-8° C. until use.

Lyophilized VD peptide (Bachem) was weighted into a glass vial and dissolved into a known volume of the Tris buffer to a concentration between 1 mg/mL and 10 mg/mL. The VD peptide was dissolved by gentle mixing and the solution was allowed to rest for between 20 and 30 minutes. The pH of the solution was checked and adjusted to 7.3 with 0.1 N sodium hydroxide. The solution was filtered through a 0.22 micron sterile filter into a sterile glass vial and stored at 2 to 8° C. until use.

The pharmacodynamic effects of VD delivered by subcutaneous infusion were investigated in a canine model high-rate paced (HRP) to a heart rate of 240 bpm (ventricular pacing) over a period of 10 days to simulate HF. The canines were divided into a control group and an experimental group. The control group received a continuous subcutaneous infusion of the Tris buffer without VD over the course of the 10 day period. The experimental group received continuous subcutaneous infusion of VD dissolved in Tris buffer at a dosing rate of 100 ng/kg·min based upon the body weight of individual canines.

Seven days prior to the beginning of HRP (Day −7), all dogs were instrumented for ventricular pacing with an IPG (implantable pulse generator) including an RA (right atrium) and RV (right ventricle) lead, and a DSI (Digital Sciences International) device in the femoral artery for arterial blood pressure monitoring. Glomerular filtration rate (GFR) was measured by iohexol clearance on the day before the beginning of HRP (Day −1). On Day 0, high-rate pacing was initiated at a rate of 240 BPM (beats per minute) and maintained continuously over the course of a 10 day period (Days 0-10). After HRP was started on Day 0, urine, blood, and hemodynamic data was collected from conscious animals to serve as a baseline.

On Days 0-10, in combination with pacing, each animal received continuous subcutaneous (SQ) infusion of an agent (Tris buffer solution for control animals and vessel dilator in Tris buffer for experimental animals) delivered via external catheter and pump. SQ infusion was performed using Medtronic MiniMed® 407C pumps equipped with 3.0 mL reservoirs (#MMT-103A) and Medtronic Silhouette® combo infusion sets (#MMT373). GFR measurements were repeated on Day 9. On Day 10 (with HRP On), a pre-term monitor was performed for urine and hemodynamic data collection on conscious animals. Once the data was collected, HRP was turned off and the animals were euthanized.

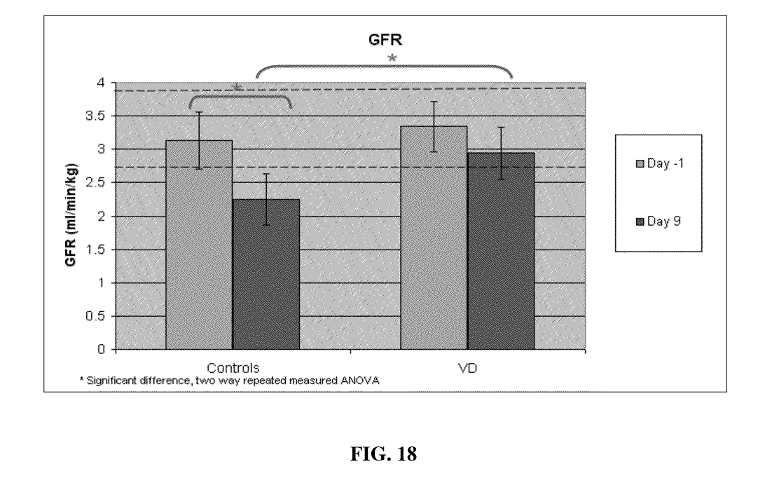

One canine died in both the experimental and control groups during the study; therefore, measurements taken on Day 0 were with 5 canines (n=5) and measurements taken on Day 10 were with 4 canines (n=4). FIG. 18 shows the results for GFR measurements taken on Day −1 and Day 9 for the control and experimental VD-treated groups. The bar graph shows the average GFR in units of mL/min per kg of body weight with the standard deviation shown by error bars.

In FIGS. 16 and 17, the observed standard deviation is indicated by error bars. As shown for the control group at Day 10, the right atrial pressure is significantly elevated in the control dogs compared with either group at Day 0 (p-value<0.05). ANOVA and post-hoc test indicates that the increase in right atrial pressure in the control group at Day 10 is statistically significant in comparison with the control and experimental groups at Day 0.

A statistically significant change in right atrial pressure is seen between the control group and the experimental group on Day 10 (p-value<0.05) in FIG. 16. An increase in right atrial pressure as a result of HRP was expected, as shown by the control group on Day 10 where mean pressure increased from 6 to 16 mmHg. Right atrial pressure increased slightly for the experimental group receiving SQ infusion of VD from Day 0 to Day 10. However, the increase observed for the experimental group is significantly less than for the control group. As such, the data presented on FIG. 16 indicates a hemodynamic benefit for SQ infusion of VD for the HF model. A decrease in the right atrial pressure is a protective cardiovascular effect.

FIG. 17 shows that pulmonary capillary wedge pressure increased as a result of HRP in both the control and experimental groups. The extent of the increase in pulmonary capillary wedge pressure in the experimental group from Day 0 to Day 10 is smaller than that observed in the control group from Day 0 to Day 10. However, the difference in pulmonary capillary wedge pressure between the control group and the experimental group on Day 10 does not appear to be statistically significant (alpha-0.05, two-way repeated measures ANOVA). The results of a statistically significant change in right atrial pressure demonstrate a measured effect on blood pressure, which can be monitored by the sensors of the present invention and used to make a decision regarding treatment using the peptides disclosed herein.

### Pharmacodynamic Study of VD in Rats

The pharmacodynamic effects of VD were investigated in a rat model. Forty male Dahl/SS rats were shipped to the animal facilities at PhysioGenix, Inc. (Milwaukee, Wis.). The rats were maintained on a low-salt diet and allowed to acclimate. After acclimation, animals had baseline parameters collected while on the low-salt diet. Baseline tail-cuff blood pressures and echocardiograms were measured. Baseline urine was collected for analysis of protein and albumin. Animals were then randomly assigned to one of 4 groups (10 animals per group):

1. Vehicle Control; low-salt diet, n=10
2. Vehicle Control; 4% salt diet, n=10
3. Vessel dilator, 100 ng/kg/min, 4% salt diet, n=10
4. Vessel dilator, 300 ng/kg/min, 4% salt diet, n=10

Lyophilized VD peptide (Bachem) was reconstituted in a Tris buffer having the same composition as the Tris buffer used in Example 7. The vehicle control groups were infused with a citrate-mannitol-saline buffer (0.66 mg/mL citric acid, 6.43 mg/mL sodium citrate, 40 mg/mL mannitol, 9 mg/mL NaCl). The animals were on a Teklad 7034 (low-salt) diet or Dyets AIN-76A 4% salt diet, as indicated, throughout a 6 week course of the study and had free access to water.

Alzet® minipumps (Durect, Corp.) were surgically implanted on Days 1, 15, and 29 of the study to maintain continuous vehicle or drug dispensing at the desired dose for a total period of 6 weeks. Urine was collected at baseline, 2, 4 and 6 weeks after the initiation of the treatment to assess proteinuria and albuminuria. After six weeks of treatment, the animals were then euthanized.

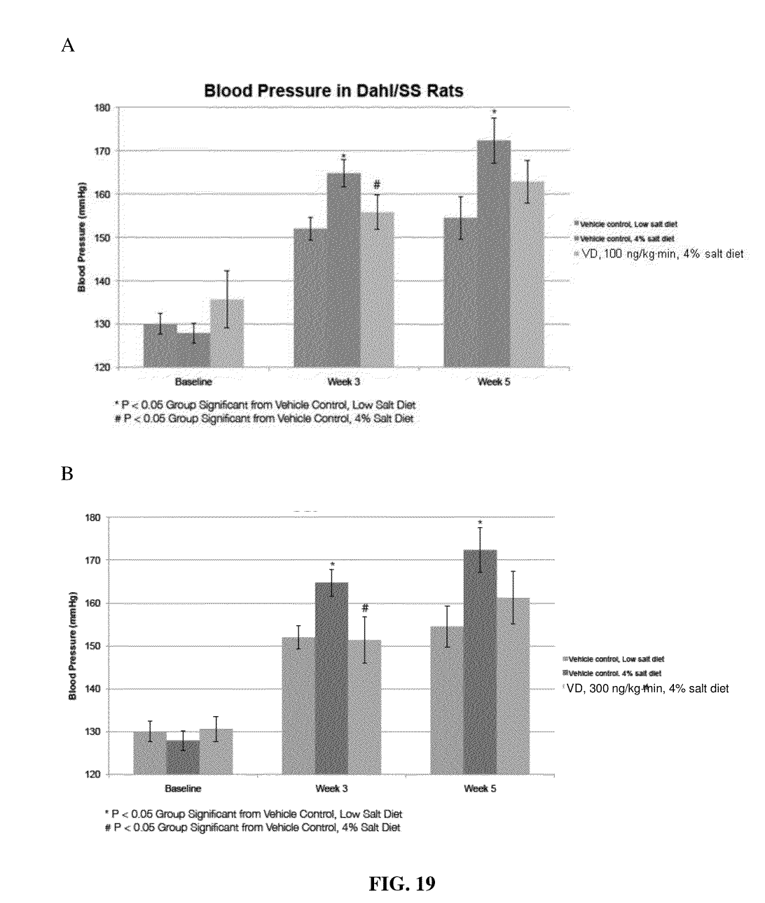

FIGS. 19 A and B present the average blood pressure for the 2 vehicle control groups on the low-salt diet and the 4% salt diet compared with either the group receiving 100 ng/kg/min of VD by SQ infusion (low-dose VD) or 300 ng/kg/min of VD (high-dose VD) by SQ infusion, respectively. Groups receiving the low-dose or high-dose of VD were maintained on the 4% salt diet. As shown in FIGS. 19 A and B, blood pressure increased in all groups. However, both the low-dose VD and the high-dose VD groups exhibited attenuated blood pressure compared with the vehicle control group on the 4% salt diet.

The vehicle control group on the 4% salt diet showed a statistically significant increase in blood pressure compared with the control group on the low-salt diet (p-value<0.05). At week 3, both the high-dose VD group and the low-dose VD group showed a statistically significant decrease in blood pressure compared with the 4% vehicle control group (p-value<0.05). The decrease in blood pressure of the high-dose VD group and the low-dose VD group at week 5 is not as statistically significant when compared with the 4% vehicle control group at week 5. Nonetheless, the groups treated with VD appear to exhibit protection against blood pressure increase induced by a high-salt diet. The standard error for all groups is shown by error bars. Reduction in blood pressure is a renal or cardiovascular effect wherein such changes can be monitored by the sensors of the present invention and used to make a decision regarding treatment using the peptides disclosed herein.

### Human Clinical Study of Subcutaneous Infusion of CD-NP Peptide

As described in U.S. Provisional application Ser. No. 13/368,225, the contents of which are incorporated herein in their entirety, Phase I clinical trials were performed in a placebo-controlled study to evaluate pharmacokinetics and pharmacodynamics of CD-NP when administered to chronic heart failure patients as a subcutaneous bolus injection or as a subcutaneous infusion. The trial was designed to understand the doses required to achieve pre-determined plasma levels of CD-NP when delivered through a subcutaneous infusion pump.

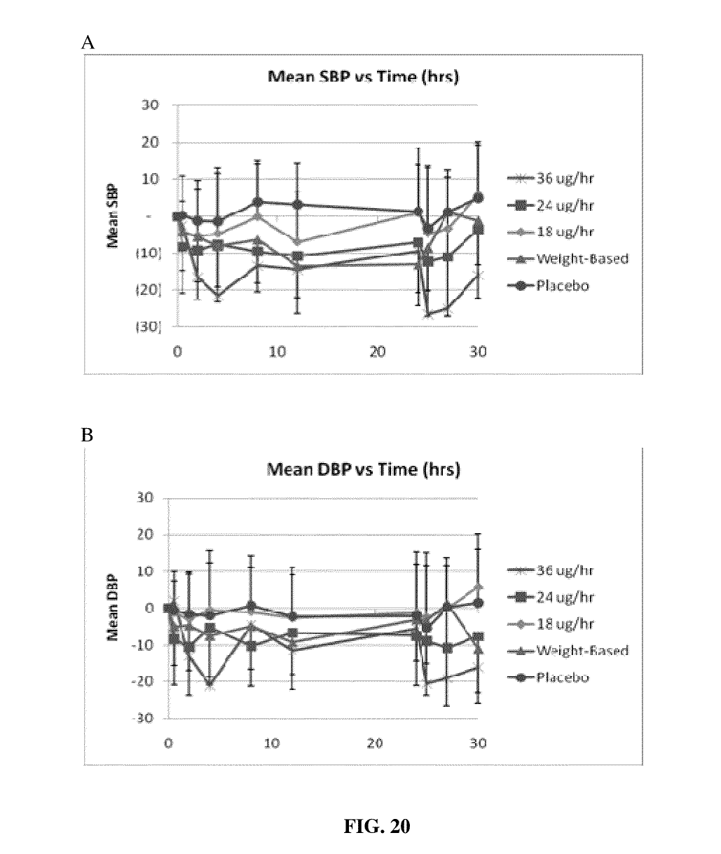

The data from the trials suggest that CD-NP infusion reduces systolic blood pressure (SBP) and diastolic blood pressure (DBP) and that the effect was observed to be larger in the high- and weight-based cohorts than the low-dose cohort. FIG. 20A shows observed mean SBP during the 24-hour infusion period including a 6-hour post-infusion period up to 30 hours from the start of infusion. FIG. 20B shows similar data for DBP. Standard error is shown in FIGS. 20A and 20B. Observed mean SBP decrease appeared to be dose dependent. During the infusion period, mean SBP decreased with CD-NP dose and gradually returned to near baseline within 3 hours. The acute post-infusion dip could have been due to a BP interaction of CD-NP with daily AM oral blood pressure medications taken by many subjects. Mean SBP values in the high-dose (24 ug/hr) and weight-based infusion cohorts were lower than baseline at all post-infusion time points through Day 7 (data not shown) with the exception of the 27 hour time point in the weight-based dosing cohort, where mean SBP was unchanged. At the Day 7 follow-up visit, mean SBP in the weight-based cohort was reduced by 10.4 mmHg (−8.0%) compared to baseline and was 2.2 mmHg (−1.7% of baseline) lower in the high-dose cohort. In the low-dose CD-NP infusion cohort at Day 7, the mean change from baseline in SBP was +3.8 mmHg.

### Pharmacodynamic Study of CD-NP in Rats

A pharmaceutical formulation of CD-NP (Nile Therapeutics, San Mateo, Calif.) was prepared. CD-NP lyophilized in a citrate-mannitol buffer (0.66 mg/mL citric acid, 6.35 mg/mL sodium citrate, 40 mg/mL mannitol) was reconstituted in sterile saline to a concentration of 3 mg/mL of the CD-NP peptide. The final composition of the pharmaceutical formulation of CD-NP was 3 mg/mL CD-NP peptide, 0.66 mg/mL citric acid, 6.35 mg/mL sodium citrate, 40 mg/mL mannitol, and 9 mg/mL sodium chloride. Chemical stability over 14 days at 37° C. in Alzet® pumps was evaluated prior to the rat study and deemed adequate.

The pharmacodynamic effects of the pharmaceutical formulation of CD-NP were investigated in a rat model. Forty male Dahl/SS rats were used to evaluate the pharmacodynamics of CD-NP. The rats were maintained on a low-salt diet and allowed to acclimate prior to the beginning of the study. After acclimation, animals had baseline parameters collected while on the low-salt diet. Baseline tail-cuff blood pressures and echocardiograms were measured. Baseline urine samples were collected for analysis of protein and albumin and baseline blood samples were collected for analysis of blood chemistries. Animals were then randomly assigned to one of 4 groups:

1. Vehicle Control; low-salt diet, n=10
2. Vehicle Control; 4% salt diet, n=10
3. High-dose CD-NP, 170 ng/kg/min CD-NP, 4% salt diet, n=10
4. Low-dose CD-NP, 85 ng/kg/min CD-NP, 4% salt diet, n=9

The vehicle control group rats were administered a citrate-mannitol-saline buffer (0.66 mg/mL citric acid, 6.43 mg/mL sodium citrate, 40 mg/mL mannitol, 9 mg/mL NaCl) without CD-NP peptide. The animals were maintained on a Teklad 7034 (low-salt) diet or Dyets AIN-76A 4% salt diet, as indicated, throughout a 6 week course of the study and had free access to water. The remaining two groups were administered the pharmaceutical formulation of CD-NP at a dosing rate or either 85 or 170 ng/kg/min, as indicated, and maintained on the 4% salt diet. The low-dose CD-NP group was limited to 9 rats due to a limited availability of CD-NP.

Alzet® minipumps were surgically implanted on Days 1, 15, and 29 of the study to maintain continuous vehicle or drug dispensing at the desired dose for a total period of 6 weeks by subcutaneous infusion. Urine was collected at baseline, 2, 4 and 6 weeks after the initiation of the treatment to assess albuminuria, creatinine clearance, electrolytes, and cGMP levels. Blood was collected at baseline, 2, 4, and 6 weeks after initiation of treatment to measure blood chemistries. Blood pressure was measured by tail cuff at baseline, 3 and 5 weeks after the start of treatment. Renal cortical blood flow was measured at week 6. Echocardiograms were performed at baseline, 2, 4, and 6 weeks after initiation of treatment to evaluate cardiac changes. After 6 weeks of treatment, the animals were then euthanized.

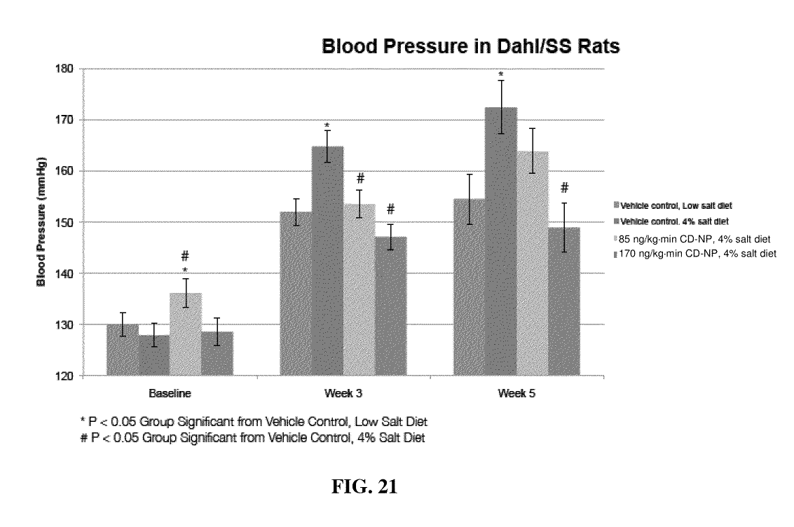

FIG. 21 presents the average blood pressure for the 2 vehicle control groups on low salt and 4% salt diet compared with the group receiving 170 ng/kg/min of CD-NP by SQ infusion and 85 ng/kg/min of CD-NP by SQ infusion. The standard error for each group is shown by error bars. As shown in FIG. 21, blood pressure increased in all groups from baseline. However, both the low-dose CD-NP and the high-dose CD-NP groups exhibited attenuated blood pressure compared with the vehicle control group on the 4% salt diet.

The vehicle control group on the 4% salt diet showed a statistically significant increase in blood pressure compared with the control group on the low-salt diet at both weeks 3 and 5 of the study (p-value<0.05). At week 3, both the high-dose CD-NP group and the low-dose CD-NP group showed a statistically significant decrease in average blood pressure compared with the 4% salt vehicle control group (p-value<0.05). The high-dose CD-NP group at week 5 showed a significantly decreased average blood pressure from the 4% salt diet vehicle control group (p-value<0.05). The decrease in average blood pressure of the low-dose CD-NP group was not as statistically significant when compared with the 4% salt vehicle control group at week 5. Nonetheless, both the high-dose CD-NP group and the low-dose CD-NP group appear to exhibit protection against blood pressure increase induced by a 4% salt diet. Reduction in blood pressure is cardiovascular protective effect wherein monitoring thereof can be used as feedback in the present invention.

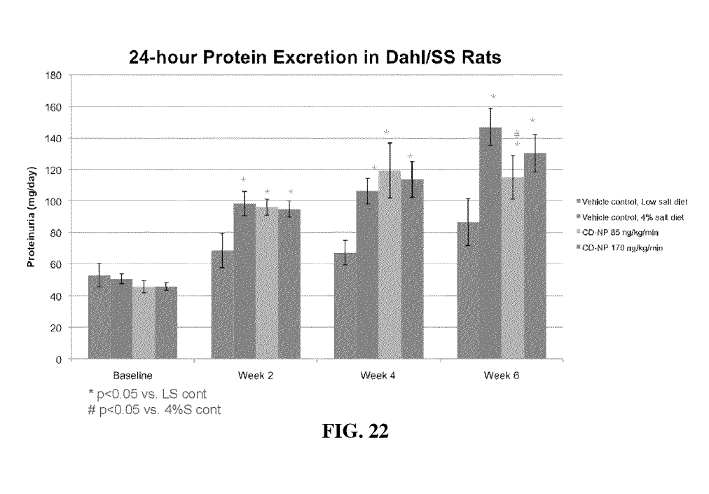

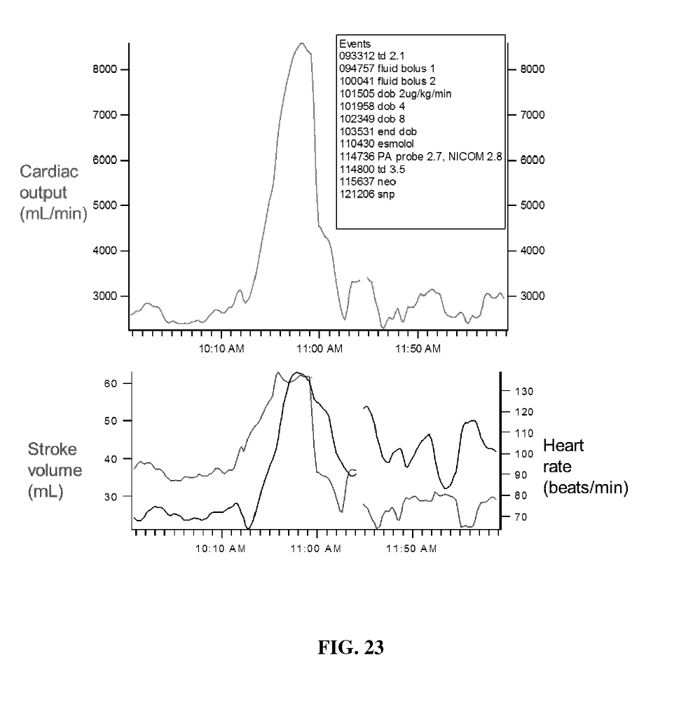

FIG. 22 shows the level of proteinuria (urine protein) in the control and experimental animal groups. Proteinuria is a measure of excess serum proteins in the urine and is an indicator of kidney dysfunction. Normal human urine does not contain any protein, although rodent urine does have low levels of secreted protein. As expected, all groups showed some proteinuria at baseline. The level of proteinuria in the CD-NP treated groups tracked with the level in the high salt diet control animals at week 2 and week 4. At week 6, the proteinuria in the high salt diet control animals continued to increase, but in both drug treated groups the level stayed steady with that measured at week 4. In addition, at week 6 the low dose CD-NP group had significantly less proteinuria than the high salt diet control group. The observed proteinuria can be monitored to provide feedback of kidney stress as contemplated by the present invention.

### NICOM Study

A noninvasive cardiac output monitoring (NICOM) monitor was utilized to measure cardiac data from an anesthetized, mechanically ventilated, 54 lb, 42 inch (nose to base of tail) male dog. FIGS. 23-27 depict the cardiac data measured by the NICOM monitor.

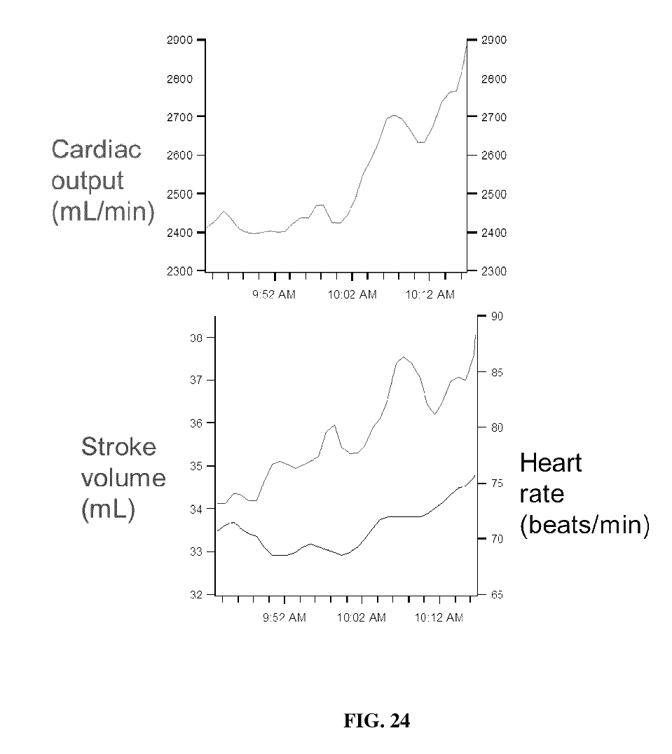

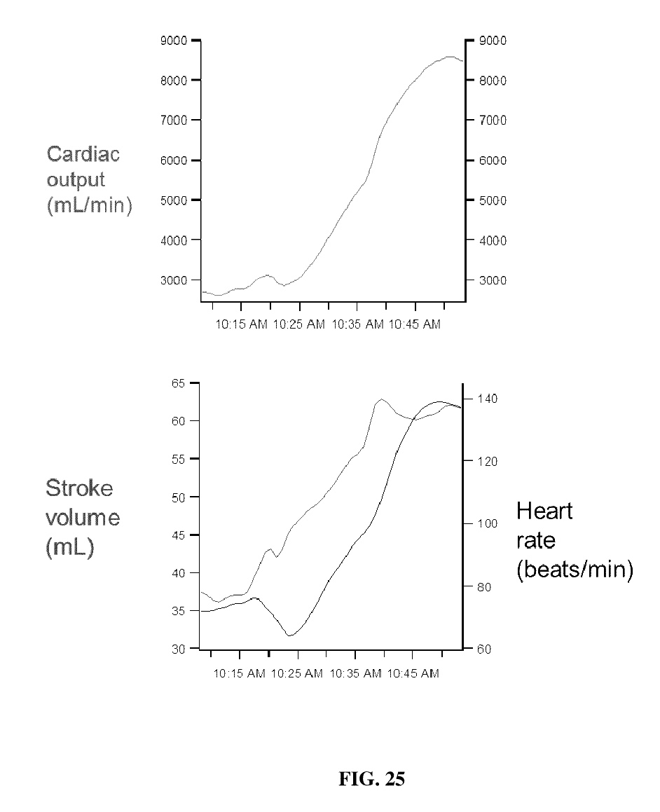

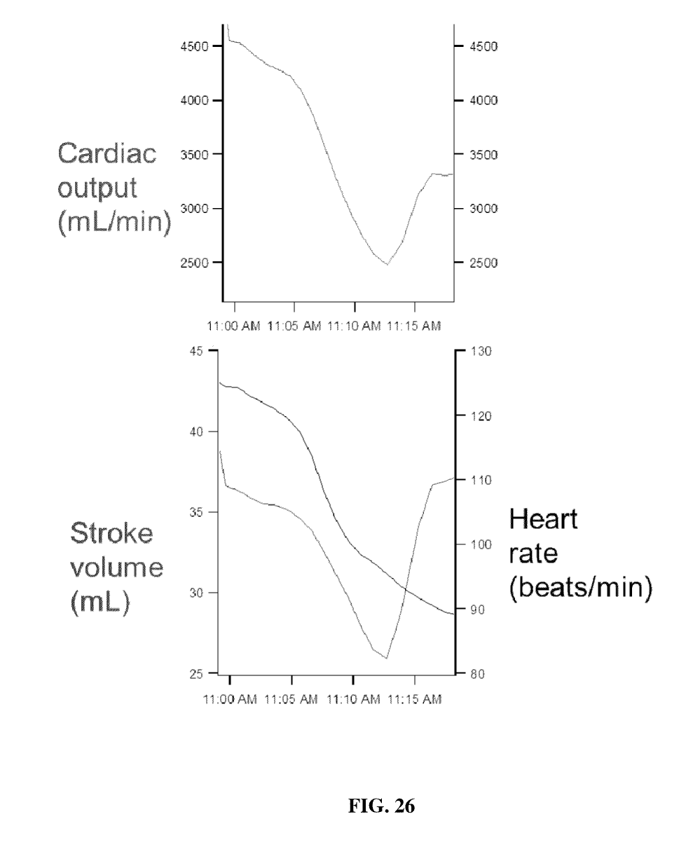

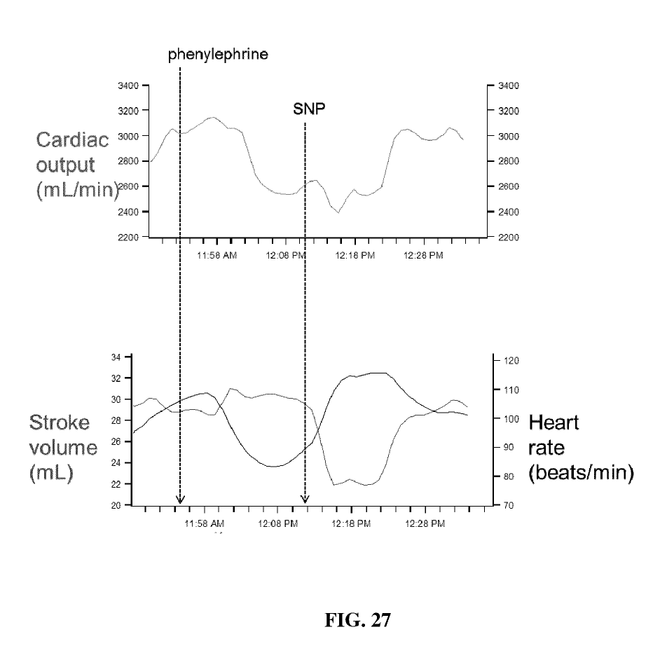

A composite of CO, SV and HR data for the entire observation period is shown in FIG. 23. The gap in data at 11:20 indicates electrocautery interference when the chest of the dog was opened. FIG. 24 depicts the data in response to sequential fluid boluses. An approximate 20% increase in CO was detected reflecting changes in both SV and HR. FIG. 25 depicts the marked increase in CO produced by incremental infusion doses of dobutamine. FIG. 26 depicts the cardiodepressant effect of the ultra-short beta-blocker esmolol given after dobutamine and recalibration. A marked decline in CO is evident reflecting changes in both SV and HR. FIG. 27 depicts the response to increasing blood pressure with phenylephrine followed by decreasing blood pressure with sodium nitroprusside (SNP). Prior to this intervention, the dog chest had been opened and commercially available flow monitors had been implanted on the pulmonary artery (PA). Baseline PA flow data, which was measured by the implantable monitors, was 2.7 L/min. The PA flow data measured by the NICOM monitor was 2.812 L/min. Phenylephrine reduced CO primarily as a consequence of reflex slowing of the HR in response to elevated blood pressure. In contrast, SNP produced a reflex increase in HR in response to a rapid drop in blood pressure that was coupled with a fall in SV as cardiac filling and ejection times were decreased. The overall effect on CO was initially modest.

Based on the data depicted in FIGS. 22-27, the NICOM monitor provides a method to measure cardiac data which is comparable to commercially available flow monitor measurements.

We claim:

1. A medical device, comprising:
one or more sensors adapted to detect at least one physiologic parameter relating to any one of cardiac function, kidney function or fluid status of a patient,
a pump for delivering one or more diuretic or natriuretic molecules to a patient, and
an algorithm for determining the need for the patient to have an increased or decreased amount of a diuretic or natriuretic molecule.

2. The medical device of claim 1, wherein the pump is controlled by a control system, the control system applying the algorithm to data received from the one or more sensors.

3. The medical device of claim 2, wherein the control system has a data aggregation device function or has access to a data aggregation device function for receiving and storing data from the one or more sensors.

4. The medical device of claim 1, wherein the one or more sensors determine a physiological parameter selected from blood pressure, pulmonary artery pressure, left atrial pressure, central venous pressure, lung fluid volume, proteinuria, plasma renin, central venous pressure, right atrial pressure, cardiac output, and glomerular filtration rate (“GFR”).

5. The medical device of claim 1, wherein the algorithm decreases a rate of administration of the pump when a physiological parameter shows a value that indicates an improvement in cardiac function, kidney function or fluid status of the patient compared to a prior value, or increases a rate of administration of the pump when a physiological parameter shows a value that indicates a worsening in cardiac function, kidney function or fluid status of the patient compared to a prior value.

6. The medical device of claim 5, wherein the algorithm stops administration by the pump when the physiological parameter improves to reach a targeted level.

7. The medical device of claim 1, wherein the algorithm issues an alert when a physiological parameter has a value that indicates a worsening in cardiac function, kidney function or fluid status of the patient and the value has reached a critical level.

8. The medical device of claim 1, wherein the algorithm can set and adjust a maximum rate of administration of the pump.

9. The medical device of claim 8, wherein the algorithm increases the maximum rate of administration of the pump by an incremental amount when all of the following are present: 1) the medical device has operated the pump at a preliminary maximum dose, where the preliminary maximum dose is a present maximum rate of administration of the pump, 2) the medical device has operated the pump at the preliminary maximum rate of administration for at least a predetermined period of time, and 3) one or more physiological parameters of the patient has been stable over the predetermined period of time to indicate that homeostatis or near homeostatis of the physiological parameter is present.

10. The medical device of claim 1, wherein the algorithm initiates the delivery of a first diuretic or a second diuretic prior to initiating delivery of the natriuretic molecule to the patient.

11. The medical device of claim 1, wherein the one or more natriuretic molecules are selected from the group consisting of long-acting natriuretic peptide (LANP), kaliuretic peptide (KP), urodilatin (URO), atrial natriuretic peptide (ANP), brain natriuretic peptide (BNP), vessel dilator (VD) and chimeric natriuretic peptides including peptides selected from CD-NP, CU-NP, and BD-NP.

12. A system, comprising:
a sensor that generates an electrical signal that varies as function of a parameter associated with least one physiologic parameter relating to any one of cardiac function, kidney function or fluid status of a patient;
a processor that processes the electrical signal to detect a change in the cardiac function, kidney function or fluid status of a patient; and
a pump controlled by the processor for administering one or more diuretic or natriuretic molecules to the patient based upon the detected change in the cardiac function, kidney function or fluid status of the patient.

13. The system of claim 12, wherein the one or more sensors determine a physiological parameter selected from blood pressure, pulmonary artery pressure, left atrial pressure, central venous pressure, lung fluid volume, proteinuria, plasma renin, central venous pressure, right atrial pressure, cardiac output, and glomerular filtration rate (“GFR”).

14. The system of claim 12, wherein a protocol for controlling the pump is adjusted in an iterative process to improve the fluid status, cardiac function or kidney function of the patient while maintaining the cardiovascular stability of the patient.

15. The system of claim 12, wherein the one or more natriuretic molecules are selected from the group consisting of long-acting natriuretic peptide (LANP), kaliuretic peptide (KP), urodilatin (URO), atrial natriuretic peptide (ANP), brain natriuretic peptide (BNP), vessel dilator (VD) and chimeric natriuretic peptides including peptides selected from CD-NP, CU-NP, and BD-NP.

16. A method, comprising the steps of:
obtaining data from one or more sensors configured to measure one or more physiological parameters of a patient;
applying an algorithm to the data received from the one or more sensors, the algorithm determining the need for the patient to have an increased or decreased amount of a diuretic or natriuretic molecules; and
performing a treatment selected from at least one of: a) administering one or more diuretic or natriuretic molecules at a rate determined by applying the algorithm, and b) stopping the administration of diuretic or natriuretic molecules as determined by applying the algorithm,
wherein the administration of the one or more diuretic or natriuretic molecules is performed by a pump for administering the diuretic or natriuretic molecules, the pump controlled by a control system for receiving data from the one or more sensors.

17. The method of claim 16, wherein the one or more physiological parameters is selected from the group consisting of blood pressure, pulmonary artery pressure, left atrial pressure, central venous pressure, lung fluid volume, proteinuria, plasma renin, central venous pressure, right atrial pressure, cardiac output, and glomerular filtration rate (“GFR”).

18. The method of claim 16, wherein a rate of administration of the diuretic or natriuretic molecules is increased if a physiological parameter measured by the one or more sensors indicates a deterioration in the fluid status, cardiac function or kidney function of the patient, or a rate of administration of the diuretic or natriuretic molecules is decreased if a physiological parameter measured by one or more sensors indicates a deterioration in the fluid status, cardiac function or kidney function of the patient.

19. The method of claim 16, wherein the data from the one or more sensors is received by a device acting as a data aggregation device for storing data received from the one or more sensors, and the data stored in the data aggregation device is accessible by the control system for controlling operation of a pump for administering the diuretic or natriuretic molecules.

20. The method of claim 16, wherein the one or more natriuretic molecules are selected from the group consisting of long-acting natriuretic peptide (LANP), kaliuretic peptide (KP), urodilatin (URO), atrial natriuretic peptide (ANP), brain natriuretic peptide (BNP), vessel dilator (VD) and chimeric natriuretic peptides including peptides selected from CD-NP, CU-NP, and BD-NP.
